(19) United States
(12) Patent Application Publication
(10) Pub. No.: US 2023/0270703 A1
(43) Pub. Date: Aug. 31, 2023

(54) COMPOUNDS AND METHODS FOR THE TREATMENT OF EYE DISORDERS

(71) Applicant: OccuRx Pty Ltd, Melbourne (AU)

(72) Inventors: Darren James Kelly, Melbourne (AU); Michelle Papadimitriou, Melbourne (AU); Chris Burns, Melbourne (AU); Eric Daniels, Melbourne (AU)

(21) Appl. No.: 18/007,714

(22) PCT Filed: Jun. 3, 2021
(86) PCT No.: PCT/US2021/035749

§ 371 Date: Dec. 1, 2022

(30) Foreign Application Priority Data
Jun. 5, 2020 (AU) 2020901856
Jun. 5, 2020 (AU) 2020901858

(51) Int. Cl. A61K 31/196

(52) U.S. Cl. CPC A61K 31/196

(57) ABSTRACT

Disclosed herein are methods of administration of (E)-2-[[3-methoxy-4- (difluoromethoxy)phenyl-l-oxo-2-propenyl]amino]benzoic acid to a subject, involving particular administration regimes for preventing, treating, reducing the severity of and/or reducing the likelihood of reoccurrence of eye conditions such as diabetic retinopathy and proliferative vitreo-retinopathy. Such eye conditions can have serious consequences, including loss of vision and, in some cases blindness.

### BACKGROUND

### Field

This disclosure relates to methods of administration of (E)-2-[[3-methoxy-4-(difluoromethoxy)phenyl-1-oxo-2-propenyl]amino]benzoic acid, involving particular administration regimes for preventing, treating, reducing the severity of and/or reducing the likelihood of reoccurrence of eye conditions, in subjects such as humans.

### Description of the Related Art

Eye disorders and diseases can have serious consequences, including loss of vision and, in some cases blindness. A number of eye disorders are associated with inflammation and/or vascular proliferation. For example, ocular disorders that have an etiology in inflammation and / or vascular proliferation include macular degeneration (age-related and non age-related). See Joussen AM, Poulaki V, et al; A central role for inflammation in the pathogenesis of diabetic retinopathy; Faseb J; 2004; 18(12): 1450-2.

Diabetic retinopathy (DR) is a common complication of diabetes and remains one of the leading causes of vision loss. See Cheung AK, Fung MK, et al; Aldose reductase deficiency prevents diabetes-induced blood-retinal barrier breakdown, apoptosis, and glial reactivation in the retina of db/db mice; Diabetes; 2005; 54(11); 3119-25; see also Santos, KG, Tschiedel B, et al; Prevalence of retinopathy in Caucasian type 2 diabetic patients from the South of Brazil and relationship with clinical and metabolic factors; Braz J Med Biol Res; 2005; 38(2); 221-5. Vision loss in DR develops by slow and progressive alterations to the retinal microvasculature (pericytes and endothelial cells) leading to breakdown of the blood-retinal barrier, pathological angiogenesis and scarring. Metabolic and biochemical changes, such as increased flux of glucose through the polyol pathway, activation of protein kinase C, oxidative damage and increased advanced glycation endproduct formations are contributors in the development of DR. Accumulating evidence indicates that vascular endothelial growth factor (VEGF) plays a critical role in angiogenesis (See Sarlos, S, Rizkalla B, et al; Retinal angiogenesis is mediated by an interaction between the angiotensin type 2 receptor, VEGF, and angiopoietin; Am J Pathol 2003; 163(3); 879-87) in DR, while intercellular adhesion molecule (ICAM-1) mediated leukocytosis results in secondary endothelial damage. See Joussen, AM, Poulaki V, et al.; Retinal vascular endothelial growth factor induces intercellular adhesion molecule-1 and endothelial nitric oxide synthase expression and initiates early diabetic retinal leukocyte adhesion in vivo; Am J Pathol; 2002; 160(2); 501-9. DR has also been recognized as a chronic inflammatory disease. With this notion, studies demonstrated that anti-inflammatory therapy prevents classic histopathological features of DR: acellular capillary formation, retinal haemorrhage development, microaneurysm progress, and pericyte loss. The current treatment for DR is laser photocoagulation, a procedure that destroys angiogenic vessels and the surrounding hypoxic tissue. See Adamis AP; Is diabetic retinopathy an inflammatory disease?; Br. J. Ophthalmol. 2002, 86(4), 363-5. Although beneficial, laser photocoagulation can destroy healthy retina, and the disease continues despite intensive treatment.

Proliferative vitreoretinopathy (PVR) is an ocular condition that occurs in about 5 to 10% of people who have had surgery to repair a retinal detachment, and occurs in up to half of people who have suffered an open globe injury. PVR is associated with proliferation of retinal pigment epithelial (RPE) and Müller cells following a trauma or break in the retina. This can trigger proliferation and migration of RPE and Müller cells as well as epithelial to mesenchymal transition of RPE cells, leading to the formation of periretinal membranes, which can be followed by contraction of the cellular membranes and traction on the retina. Retinal detachment patients that suffer from PVR have an increased likelihood of having a further retinal detachment in the same eye. PVR can lead to serious consequences including vision loss and blindness. See Friedlander; Fibrosis and diseases of the eye; J Clin Invest.; 2007; 117(3); 576-586; Guidry C, Proliferative Vitreoretinopathy, Chapter 78, in Ocular Disease, Mechanism and Management; 2010; 612-7. Other ocular conditions associated with such pathologies include abnormal eye wound healing following eye trauma, or abnormal healing following eye surgery.

It would be desirable to provide further therapies for the treatment and/or prevention of eye conditions. However, the development of such new therapies is an extremely lengthy, challenging and expensive task, involving extensive preclinical and clinical testing, with many agents failing for a variety of reasons, including for example poor pharmacokinetic properties, such as poor bioavailability, rapid metabolism, and/or an inability to dose the active agent in a manner which provide an in vivo concentration that is therapeutically effective for a sufficient period of time. It is also important that, upon administration of an active compound, the compound reaches the target tissue or organ sufficiently quickly to provide therapeutic effects. For example, the blood retinal barrier (BRB) is composed of two types of cells, retinal capillary endothelial cells and retinal pigment epithelial cells. Both can be an impediment to drug delivery. See Varela-Fernandez R, Diaz-Tome V, et al; Drug Delivery to the Posterior Segment of the Eye: Biopharmaceutic and Pharmacokinetic Considerations; Pharmaceutics; 2020; 12(3); 269.

In the field of small molecule therapeutics, factors such as log P, log D, the number of hydrogen bond donors and acceptors, and the presence of acidic or basic groups can all play a significant role in affecting compound properties. For example, compounds with strongly acidic or basic groups tend to be poorly absorbed, and neutral molecules are more readily able to traverse lipid membranes than charged molecules. See Manallack et al; The significance of acid/base properties in drug discovery; Chem Soc Rev; 2013; 42 (2); 485-496.

Physicochemical properties are also important for delivery of drugs across the blood retinal barrier. For example, in the retinal pigment epithelial cells, drug permeability can depend on factors such as molecular weight, lipophilicity and protein binding. See Klein R; Klein BE, et al; The relation of retinal vessel caliber to the incidence and progression of diabetic retinopathy: XIX: the Wisconsin Epidemiologic Study of Diabetic Retinopathy; Arch Ophthalmol; 2004; 122(1); 76-83.

An important parameter for any drug is its in vivo half-life, which is the time taken for the concentration of drug in the plasma to decrease by 50%. At the very least, biologically active compounds with a short half-life need to be dosed more frequently, which can lead to issues with patient compliance. Further, many compounds with short half-lives are ultimately not suitable for use as therapeutic agents due to the inability to provide therapeutically effective concentrations for a useful time period, or due to the need to dose such high amounts of compound that significant side effects result.

Half-life is related to the volume of distribution of a compound (the ability of a compound to distribute from the systemic circulation into tissue), and to its clearance (how rapidly the compound is metabolised). Binding of compounds to blood plasma proteins has a significant effect on volume of distribution, with acidic compounds having a greater tendency to be highly plasma protein-bound and to have a lower volume of distribution than other charge types, which can contribute to a shorter half-life.

As discussed above, there remains a need for effective therapies for treating eye conditions, for example those associated with inflammation or vascular proliferation such as diabetic retinopathy and age-related macular degeneration, or those associated with non-vascular proliferation, epithelial to mesenchymal transition and/or tissue contraction, such as proliferative vitreoretinopathy. There is a particular need for therapies which have pharmacokinetic properties such that they achieve therapeutically relevant concentrations in the eye for a sustained period of time, which allow for infrequent dosing, and which have sufficient oral bioavailability to allow for administration by the oral route.

### SUMMARY

It has been found that the compound (E)-2-[[3-methoxy-4-(difluoro methoxy)phenyl-1-oxo-2-propenyl]amino]benzoic acid

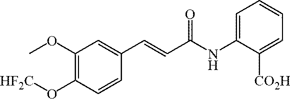

is a pharmaceutically active agent with surprisingly advantageous pharmacokinetic properties. It has been found that the compound has an unexpectedly long half-life in humans, and that sustained therapeutically effective concentrations of the compound can be achieved in vivo following oral dosing, facilitating infrequent dosing. Further, experiments in animals show that significant concentrations of the compound reach the eye quickly after oral dosing. Accordingly, the compound is of particular utility in treating ocular conditions, for example those associated with inflammation and/or vascular proliferation.

Accordingly, in some embodiments, provided herein is a method of preventing, treating, reducing the severity of and/or reducing the likelihood of reoccurrence of an eye condition in a subject, comprising,
administering a compound of formula (I):

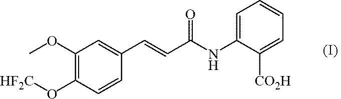

or a pharmaceutically acceptable salt thereof, to the subject,
wherein the compound or pharmaceutically acceptable salt thereof is administered at a frequency selected from once per day and twice per day.

In some embodiments, the compound or pharmaceutically acceptable salt thereof is administered orally.

In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the compound or pharmaceutically acceptable salt thereof is administered twice per day. In some embodiments, the compound or pharmaceutically acceptable salt thereof is administered once per day.

In some embodiments, the subject is a human.

Also provided herein is a method of preventing, treating, reducing the severity of and/or reducing the likelihood of reoccurrence of an eye condition in a subject, comprising,
administering a pharmaceutical composition comprising i) a compound of formula (I):

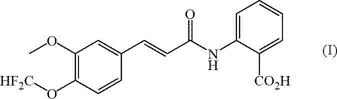

or a pharmaceutically acceptable salt thereof, and ii) a pharmaceutically acceptable excipient, to the subject,
wherein the pharmaceutical composition is administered at a frequency selected from once per day and twice per day.

In some embodiments, the pharmaceutical composition is administered orally. In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the pharmaceutical composition is administered twice per day. In some embodiments, the pharmaceutical composition is administered once per day.

In some embodiments, the subject is a human.

In some embodiments, the eye condition is an eye condition associated with at least one of inflammation and vascular proliferation. In some embodiments, the eye condition is selected from the group consisting of diabetic retinopathy and age-related macular degeneration. In some embodiments, the eye condition is proliferative diabetic retinopathy. In some embodiments, the eye condition is non-proliferative diabetic retinopathy. In some embodiments, the eye condition is wet age-related macular degeneration.

In some embodiments, the eye condition is an eye condition which is associated with at least one of the following: non-vascular cell proliferation, epithelial to mesenchymal transition, and tissue contraction. In some embodiments, the eye condition is proliferative vitreoretinopathy.

In some embodiments, the method is for preventing or reducing the likelihood of reoccurrence of a further retinal detachment, in a subject who has previously experienced a retinal detachment.

Also provided herein is the use of a compound of formula (I):

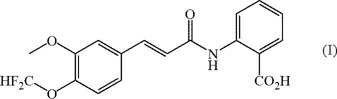

or a pharmaceutically acceptable salt thereof,
for the manufacture of a medicament for the prevention, treatment, reduction of severity of and/or reduction of likelihood of reoccurrence of an eye condition in a subject,
wherein the compound or pharmaceutically acceptable salt thereof is administered to the subject at a frequency selected from once per day and twice per day.

Also provided herein is a compound of formula (I):

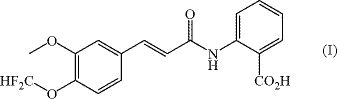

or a pharmaceutically acceptable salt thereof, for use in the prevention, treatment, reduction of severity of and/or reduction of likelihood of reoccurrence of an eye condition in a subject, wherein the compound or pharmaceutically acceptable salt thereof is administered to the subject at a frequency selected from once per day and twice per day.

Also provided herein is a pharmaceutical composition comprising i) a compound of formula (I):

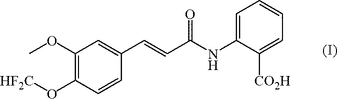

or a pharmaceutically acceptable salt thereof, and ii) a pharmaceutically acceptable excipient, for use in the prevention, treatment, reduction of severity of and/or reduction of likelihood or reoccurrence of an eye condition in a subject, wherein the pharmaceutical composition is administered to the subject at a frequency selected from once per day and twice per day.

In some embodiments, following oral administration, the compound has a terminal half-life in the range of from 18 to 30 hours. In some embodiments, following oral administration, the compound has a terminal half-life in the range of from 18 to 24 hours.

Also provided herein is a method of preventing, treating, reducing the severity of and/or reducing the likelihood of reoccurrence of an eye condition in a subject, comprising,
administering a compound of formula (I):

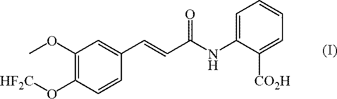

or a pharmaceutically acceptable salt thereof, to the subject,
wherein the amount of compound administered is in the range of from 5 mg to 250 mg per day, or the amount of pharmaceutically acceptable salt administered provides an amount of compound in the range of from 5 mg to 250 mg per day.

In some embodiments, the compound or pharmaceutically acceptable salt thereof is administered orally. In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the amount of compound administered is in the range of from 100 mg to 250 mg per day, or the amount of pharmaceutically acceptable salt administered provides an amount of compound in the range of from 100 mg to 250 mg per day.

In some embodiments, the subject is a human.

Also provided herein is a method of preventing, treating, reducing the severity of and/or reducing the likelihood of reoccurrence of an eye condition in a subject, comprising,
administering a pharmaceutical composition comprising i) a compound of formula (I):

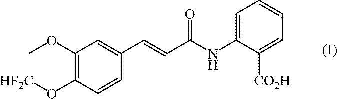

or a pharmaceutically acceptable salt thereof, and ii) a pharmaceutically acceptable excipient, to the subject,
wherein the amount of pharmaceutical composition administered provides an amount of compound in the range of from 5 mg to 250 mg per day.

In some embodiments, the pharmaceutical composition is administered orally. In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the amount of pharmaceutical composition administered provides an amount of compound in the range of from 100 mg to 250 mg per day.

In some embodiments, the subject is a human.

In some embodiments, the eye condition is an eye condition associated with one or more of inflammation and vascular proliferation. In some embodiments, the eye condition is selected from the group consisting of diabetic retinopathy and age-related macular degeneration. In some embodiments, the eye condition is proliferative diabetic retinopathy. In some embodiments, the eye condition is non-proliferative diabetic retinopathy. In some embodiments, the eye condition is wet age-related macular degeneration.

In some embodiments, the eye condition is an eye condition which is associated with at least one of non-vascular cell proliferation, epithelial to mesenchymal transition and tissue contraction. In some embodiments, the eye condition is proliferative vitreoretinopathy.

In some embodiments, the method is for preventing or reducing the likelihood of reoccurrence of a further retinal detachment, in a subject who has previously experienced a retinal detachment.

Also provided herein is the use of a compound of formula (I):

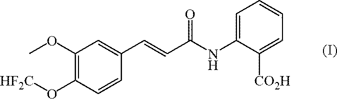

or a pharmaceutically acceptable salt thereof,
for the manufacture of a medicament for the prevention, treatment, reduction of severity of and/or reduction of likelihood of reoccurrence of an eye condition in a subject,
wherein the amount of compound administered is in the range of from 5 mg to 250 mg per day, or the amount of pharmaceutically acceptable salt administered provides an amount of compound in the range of from 5 mg to 250 mg per day.

There is also provided a compound of formula (I):

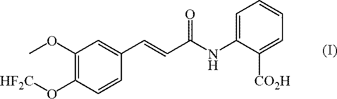

or a pharmaceutically acceptable salt thereof,
for use in the prevention, treatment, reduction of severity of and/or reduction of likelihood of reoccurrence of an eye condition in a subject,
wherein the amount of compound administered is in the range of from 5 mg to 250 mg per day, or the amount of pharmaceutically acceptable salt administered provides an amount of compound in the range of from 5 mg to 250 mg per day.

Also provided herein is a pharmaceutical composition comprising i) a compound of formula (I):

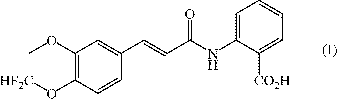

or a pharmaceutically acceptable salt thereof, and ii) a pharmaceutically acceptable excipient, for use in the prevention, treatment, reduction of severity of and/or reduction of likelihood of reoccurrence of an eye condition in a subject,
wherein the amount of pharmaceutical composition administered provides an amount of compound in the range of from 5 mg to 250 mg per day.

In some embodiments, following oral administration, the compound has a terminal half-life in the range of from 18 to 30 hours. In some embodiments, following oral administration, the compound has a terminal half-life in the range of from 18 to 24 hours.

Also provided herein is a method of preventing, treating, reducing the severity of and/or reducing the likelihood of reoccurrence of an eye condition in a subject, comprising,
administering a first dose or series of first doses of a compound of formula (I):

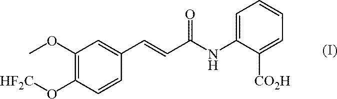

or a pharmaceutically acceptable salt thereof, to the subject, and
subsequently administering a second dose or series of second doses of the compound of formula (I) or pharmaceutically acceptable salt thereof, to the subject,
wherein the amount of compound or pharmaceutically acceptable salt in the or each first dose is greater than the amount of compound or pharmaceutically acceptable salt in the or each second dose.

In some embodiments, the compound or pharmaceutically acceptable salt thereof is administered orally. In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the subject is a human.

Also provided herein is a method of preventing, treating, reducing the severity of and/or reducing the likelihood of reoccurrence of an eye condition in a subject, comprising,
administering a first dose or series of first doses of a pharmaceutical composition comprising a) a compound of formula (I):

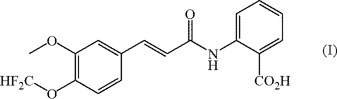

or a pharmaceutically acceptable salt thereof, and b) a pharmaceutically acceptable excipient, to the subject, and
subsequently administering a second dose or series of second doses of a pharmaceutical composition comprising a) the compound of formula (I) or a pharmaceutically acceptable salt thereof, and b) a pharmaceutically acceptable excipient, to the subject,
wherein the amount of compound or pharmaceutically acceptable salt in the or each first dose is greater than the amount of compound or pharmaceutically acceptable salt in the or each second dose.

In some embodiments, the pharmaceutical composition is administered orally. In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the subject is a human.

In some embodiments, the eye condition is an eye condition associated with one or more of inflammation and vascular proliferation. In some embodiments, the eye condition is selected from the group consisting of diabetic retinopathy and age-related macular degeneration. In some embodiments, the eye condition is proliferative diabetic retinopathy. In some embodiments, the eye condition is non-proliferative diabetic retinopathy. In some embodiments, the eye condition is wet age-related macular degeneration.

In some embodiments, the eye condition is associated with one or more of non-vascular cell proliferation, epithelial to mesenchymal transition, and tissue contraction. In some embodiments, the eye condition is proliferative vitreoretinopathy.

In some embodiments, the method is for preventing or reducing the likelihood of reoccurrence of a further retinal detachment, in a subject who has previously experienced a retinal detachment.

Also provided herein is the use of a compound of formula (I):

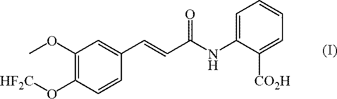

or a pharmaceutically acceptable salt thereof,
for the manufacture of a medicament for the prevention, treatment, reduction of severity of and/or reduction of likelihood of reoccurrence of an eye condition in a subject,
wherein a first dose or series of first doses of the compound of formula (I) or a pharmaceutically acceptable salt thereof, is administered to the subject, and
subsequently a second dose or series of second doses of the compound of formula (I) or pharmaceutically acceptable salt thereof, is administered to the subject,
and wherein the amount of compound or pharmaceutically acceptable salt in the or each first dose is greater than the amount of compound or pharmaceutically acceptable salt in the or each second dose.

There is also provided a compound of formula (I):

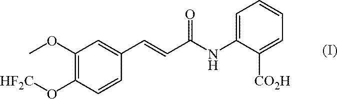

or a pharmaceutically acceptable salt thereof,
for use in the prevention, treatment, reduction of severity of and/or reduction of likelihood of reoccurrence of an eye condition in a subject,
wherein a first dose or series of first doses of the compound of formula (I) or a pharmaceutically acceptable salt thereof, is administered to the subject, and
subsequently a second dose or series of second doses of the compound of formula (I) or pharmaceutically acceptable salt thereof, is administered to the subject,
and wherein the amount of compound or pharmaceutically acceptable salt in the or each first dose is greater than the amount of compound or pharmaceutically acceptable salt in the or each second dose.

Also provided herein is a pharmaceutical composition comprising i) a compound of formula (I):

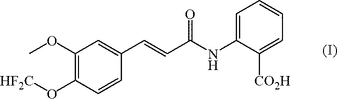

or a pharmaceutically acceptable salt thereof, and ii) a pharmaceutically acceptable excipient, for use in the prevention, treatment, reduction of severity of and/or reduction of likelihood of reoccurrence of an eye condition in a subject,
wherein a first dose or series of first doses of a pharmaceutical composition comprising i) and ii) is administered to the subject, and
a subsequently a second dose or series of second doses of a pharmaceutical composition comprising i) and ii) is administered to the subject,
and wherein the amount of compound or pharmaceutically acceptable salt in the or each first dose is greater than the amount of compound or pharmaceutically acceptable salt in the or each second dose.

In some embodiments, following oral administration, the compound has a terminal half-life in the range of from 18 to 30 hours. In some embodiments, following oral administration, the compound has a terminal half-life in the range of from 18 to 24 hours.

### BRIEF DESCRIPTION OF THE DRAWINGS

FIG. 1 is a graph showing rapid absorption and long half-life of (E)-2-[[3-methoxy-4-(difluoromethoxy)phenyl-l-oxo-2-propenyl]amino]benzoic acid in humans. The figure shows the plasma pharmacokinetic profile of healthy male volunteers after oral administration of a single dose of 50 mg of (E)-2-[[3-methoxy-4-(difluoromethoxy)phenyl-1-oxo-2-propenyl]amino]benzoic acid. Data points represent the mean of six males and lines represent the standard deviation.

FIG. 2 graphically depicts data, demonstrating that rapid distribution of (E)-2-[[3-methoxy-4-(difluoromethoxy)phenyl-1-oxo-2-propenyl]amino]benzoic acid to the retina in rats. The figure shows the pharmacokinetic profile of plasma and retina of male SD rats after oral administration of a single dose of 50 mg/kg of (E)-2-[[3-methoxy-4-(difluoromethoxy)phenyl-1-oxo-2-propenyl]amino]benzoic acid. Data points represent the mean of 3 animals and lines represent the standard deviation.

### DETAILED DESCRIPTION

### General Definitions

The following definitions apply to the terms as used throughout this specification, unless otherwise limited in specific instances.

Unless specifically defined otherwise, all technical and scientific terms used herein shall be taken to have the same meaning as commonly understood by one of ordinary skill in the art (e.g., chemistry, medicinal chemistry and the like).

As used herein, the term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/-20%, more preferably +/- 10%, and refers to +/- 5%, more preferably, of the designated value.

As used herein, singular forms “a”, “an” and “the” include plural aspects, unless the context clearly indicates otherwise.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Compound of Formula (I)

The present disclosure relates to the compound of formula (I) and salts thereof. The compound of formula (I) is (E)-2-[[3-methoxy-4-(difluoromethoxy)phenyl-1-oxo-2-propenyl]amino]benzoic acid, and has the structure:

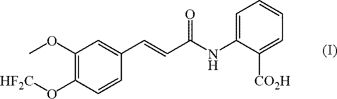

A group of cinnamoyl anthranilic acid derivatives, including compound (I), which is disclosed in WO2009/079692A1 (the entire contents of which are incorporated herein by reference). WO2012/068612A1 (the entire contents of which are incorporated herein by reference) also discloses such compounds for use in therapy of certain eye disorders.

As discussed above, it has now been found that compound (I) has unexpected and surprising pharmacokinetic properties, such that it achieves a long in vivo half-life in humans, has oral bioavailability sufficient for oral dosing, and reaches the eye quickly following administration. This allows for convenient and infrequent dosing to patients, and facilitates high levels of patient compliance and better medical outcomes.

The compound is also understood to be particularly effective for treating eye disorders including non-proliferative diabetic retinopathy, and preventing or slowing its progression to proliferative diabetic retinopathy. In vitro and in vivo pre-clinical studies have demonstrated that the compound inhibits processes that contribute to scar formation, growth of new and abnormal blood vessels, and inflammation in the retina. The compound does so by inhibiting multiple signalling pathways that are activated in retinal diseases.

The compound of formula (I) may be used as the free acid, or in salt form. Suitable salts of the compound of formula (I) include those formed with organic or inorganic bases. As used herein, the phrase “pharmaceutically acceptable salt” refers to pharmaceutically acceptable organic or inorganic salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl -propylamine, amino acids, for example lysine or arginine, or a mono-, di- or trihydroxy lower alkylamine, for example mono-, di- or triethanolamine. The counterion may be any organic or inorganic moiety that stabilizes a charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compound of formula (I) and salts thereof.

### Compound Synthesis

The compound of formula (I) may be prepared by any suitable synthetic route.

In some embodiments, the compound is prepared by reaction of 4-(difluoromethoxy)-3-methoxybenzaldehyde with 2-[(carboxyacetyl)amino]benzoic acid, e.g. in the presence of a suitable amine such as piperidine and in a solvent such as toluene, at elevated temperature and with removal of water

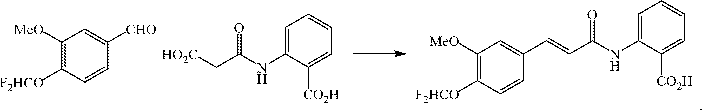

In such a process, the starting benzaldehyde(4-(difluoromethoxy)-3-methoxybenzaldehyde) may for example be prepared by reaction of methyl chlorodifluoroacetate with vanillin, e.g. in the presence of a base such as potassium carbonate and a solvent such as DMF, and at elevated temperature.

The starting benzoic acid (2-[(carboxyacetyl)amino]benzoic acid) may for example be prepared by reaction of anthranilic acid with Meldrum’s acid, for example in a solvent such as toluene, at elevated temperature and removal of water.

### Eye Conditions

The compound of formula (I) finds use in therapy of eye conditions. The compound of formula (I) may be used for prevention, treatment, reduction of severity of and/or reduction of likelihood of reoccurrence of such conditions.

In some embodiments, the compound of formula (I) or salt or pharmaceutical composition is used for the treatment of eye conditions. As used herein, the term “treating” includes lessening of the signs, symptoms or causes associated with a specific disorder or condition and eliminating said signs, symptoms or causes. It also includes slowing of or stopping the progression of a specific disease or condition. For example, as used herein, the term “treating diabetic retinopathy” includes lessening of symptoms associated with diabetic retinopathy, and includes slowing of or stopping the progression of diabetic retinopathy.

In some embodiments, the compound of formula (I) is used for the prevention of eye conditions. As used herein, the term “prevention” includes prophylaxis of the specific disorder or condition, and prevention or reduction of the onset of one or more symptoms associated with the disorder or condition. For example, as used herein, the term “preventing diabetic retinopathy” includes preventing the onset of the symptoms associated with diabetic retinopathy.

In some embodiments, the eye condition is an eye condition associated with either inflammation or vascular proliferation. In some embodiments, the eye condition is an eye condition associated with both inflammation and vascular proliferation.

In some embodiments, the eye condition is selected from the group consisting of diabetic retinopathy, macular degeneration, macular edema, corneal edema, anterior and posterior uveitis, pterygium, corneal disease, dry eye, conjunctivitis, allergy- and laser-induced exudation and ocular von Hippel-Lindau disease.

In some embodiments, the eye condition is a corneal disease caused by infection from a microbe or microorganism. The microbe or microorganism may for example be a bacteria, virus, fungi, amoebas or a parasite.

In some preferred embodiments, the eye condition is diabetic retinopathy (DR). Based on the extent of vascular abnormalities, DR can be broadly categorized into non-proliferative DR (NPDR) and proliferative DR (PDR) (11).

In NPDR, hyperglycaemia induces thickening of capillary basement membrane, apoptosis or ‘dropout’ of pericytes, microaneurysms and vascular leakage. Blockade of retinal capillaries causes localized hypoxia, which increases the production of angiogenic growth factors. In some microvessels, endothelial cells become apoptotic resulting in acellular capillaries (devoid of both pericytes and endothelial cells), capillary closure and areas of retinal non-perfusion. Adherent leukocytes may also contribute to the lesion by causing retinal capillary occlusion (1). Multiple haemorrhages, soft exudates, cotton wool spots, intraretinal microvascular abnormalities and venous beading and loops develop. Increased areas of tissue non-perfusion stimulate the production of angiogenic factors leading to the proliferation of vessels, which is the hallmark feature of PDR.

There is a need for therapies to treat non-proliferative diabetic retinopathy. For example, with NPDR, at present in the early stages regular monitoring may be the only option (https://www.aoa.org/patients-and-public/eye-and-vision-problems/glossary-of-eye-and-vision-conditions/diabetic-retinopathy). Thus, in some embodiments, the compound of formula (I) or pharmaceutically acceptable salt or pharmaceutical composition is used for the prevention or treatment of NPDR. For example, it may be used to prevent or slow progression to proliferative diabetic retinopathy.

In some embodiments, the compound of formula (I) or pharmaceutically acceptable salt or pharmaceutical composition is used for the prevention or treatment of proliferative diabetic retinopathy.

In some preferred embodiments, the eye condition is macular degeneration. In some embodiments, the compound of formula (I) or pharmaceutically acceptable salt or pharmaceutical composition is used for the prevention or treatment of age-related macular degeneration. In some embodiments, the compound of formula (I) or pharmaceutically acceptable salt or pharmaceutical composition is used for the prevention or treatment of non age-related macular degeneration. In some embodiments, the compound of formula (I) or pharmaceutically acceptable salt or pharmaceutical composition is used for the prevention or treatment of wet age-related macular degeneration. In some embodiments, the compound of formula (I) or pharmaceutically acceptable salt or pharmaceutical composition is used for the prevention or treatment of dry age-related macular degeneration.

In some embodiments, the compound of formula (I) or pharmaceutically acceptable salt thereof, or pharmaceutical composition, finds use in therapy of eye conditions associated with non-vascular cell proliferation, epithelial to mesenchymal transition, and/or tissue contraction. Such conditions can have severe effects including loss of vision and blindness.

Such pathologies are characteristic of conditions such as proliferative vitreoretinopathy. One or more of those pathologies is also prevalent in conditions such as complications arising from eye wound healing, complications arising from eye surgery, complications arising from retinal phototherapy, or a condition such as scarring of the eye. Subjects that have proliferative retinopathy following surgery to repair a retinal detachment are often at greater risk of retinal detachment, as the formation of membranes and tissue traction can place the retina under strain.

Conditions for which the compound of formula (I) or pharmaceutically acceptable salt thereof provides an effective therapy, include those associated with one or more of non-vascular cell proliferation, epithelial to mesenchymal transition, and/or tissue contraction. In other words, in such conditions, one or more of non-vascular cell proliferation, epithelial to mesenchymal transition, and/or tissue contraction may be observed to occur or have occurred in a subject.

In some embodiments, the eye condition is an eye condition associated with non-vascular cell proliferation. For example, it may be an eye condition associated with retinal pigment epithelial (RPE) cell proliferation and/or Müller cell proliferation. In some embodiments, the eye condition is associated with RPE cell proliferation. In some embodiments, the eye condition is associated with Müller cell proliferation.

In some embodiments, the compound of formula (I) or pharmaceutically acceptable salt thereof, or pharmaceutical composition comprising the compound or salt, inhibits non-vascular cell proliferation, for example, inhibits RPE cell proliferation and/or Müller cell proliferation.

In some embodiments, the compound or pharmaceutically acceptable salt thereof prevents and/or reduces proliferation and/or migration of cells following a retinal break, tear, detachment, phototherapy, or following eye trauma.

In some embodiments, the eye condition is associated with epithelial to mesenchymal transition (EMT). Epithelial-mesenchymal transition is a process by which polarised epithelial cells undergo biochemical changes, gaining migratory and invasive properties to assume a mesenchymal phenotype. EMT has been shown to occur in processes including wound healing.

In some embodiments, the eye condition is associated with epithelial to mesenchymal transition of retinal pigment epithelial cells.

In some embodiments, the compound of formula (I) or pharmaceutically acceptable salt thereof, or pharmaceutical composition comprising the compound or salt, inhibits epithelial to mesenchymal transition, for example inhibits epithelial to mesenchymal transition of retinal pigment epithelial cells.

In some embodiments, the eye condition is associated with tissue contraction. Contraction of periretinal membrane tissue occurs in conditions such as proliferative vitreoretinopathy, and can cause retinal detachment.

In some embodiments, the compound of formula (I) or pharmaceutically acceptable salt thereof, or pharmaceutical composition comprising the compound or salt, inhibits tissue contraction, for example inhibits contraction of periretinal membrane tissue.

In some embodiments, the compound of formula (I) or pharmaceutically acceptable salt thereof, or pharmaceutical composition comprising the compound or salt, prevents and/or reduces retinal traction.

In some embodiments, the compound of formula (I) or pharmaceutically acceptable salt thereof, or pharmaceutical composition comprising the compound or salt, prevents and/or reduces cell sheet and/or membrane formation.

In some embodiments, the eye condition is an eye condition resulting from a retinal detachment.

In some embodiments, the eye condition is an eye condition resulting from eye surgery. For example, the action of carrying out eye surgery to treat an eye disorder or eye injury may itself lead to excessive wound healing processes, formation of scar tissue and/or further vision problems.

In some embodiments, the eye condition is an eye condition associated with wound healing. In some embodiments, the condition is proliferative vitreoretinopathy (PVR). PVR is a disease which can develop as a complication of rhegmatogenous retinal detachment and associated surgery to correct retinal detachment. PVR is associated with migration and proliferation of cells following a break in the retina or trauma, leading to formation of membranes in the peri-retinal area, followed by contraction of the membranes and traction on the retina that can cause retinal detachment. Proliferative vitreoretinopathy may also be referred to as massive vitreous retraction or massive periretinal proliferation. Factors affecting the likelihood of PVR include existence of PVR prior to surgery, duration of retinal detachment before surgery takes place, size of retinal tear, existence of intraocular inflammation, vitreous haemorrhage, and trauma to the eye. PVR has been described as being the biggest obstacle to successful retinal reattachment surgery). See Spirn and Regillo; Proliferative Retinopathy; Retinal Physician; Jan/February 2008;

PVR is graded as Grade A, B or C by the Silicone Oil Study, and as Grade A, B, C or D by the Retina Society Terminology Committee. In some embodiments, the PVR is Grade A as graded by the Silicone Oil Study. In some embodiments, the PVR is Grade B as graded by the Silicone Oil Study. In some embodiments, the PVR is Grade C as graded by the Silicone Oil Study. In some embodiments, the PVR is Grade A as graded by the Retina Society Terminology Committee. In some embodiments, the PVR is Grade B as graded by the Retina Society Terminology Committee. In some embodiments, the PVR is Grade C as graded by the Retina Society Terminology Committee. In some embodiments, the PVR is Grade D as graded by the Retina Society Terminology Committee. See Di Lauro S, Kadhim MR, Charteris DG and Pastor JC, Classifications for proliferative vitreoretinopathy (PVR): An analysis of their use in publications over the last 15 years; J Ophthalmol; 2016; 2016; 7807596

In some embodiments, the condition is an eye condition associated with eye trauma. For example, the condition may be an eye condition associated with a globe trauma.

In some embodiments, the condition is a complication associated with retinal phototherapy, such as a complication associated with pan-retinal photocoagulation. Retinal phototherapy such as panretinal photocoagulation therapy can be used to treat conditions such as retinal ischemia, retinal neovascularisation and proliferative diabetic retinopathy. However, the use of such therapies can lead to complications including retinal scarring and vision loss. The compound of formula (I) or pharmaceutically acceptable salt thereof, or pharmaceutical composition comprising the compound or salt, finds use in preventing or reducing the severity of such complications.

In some embodiments, the complication associated with retinal phototherapy (e.g. pan-retinal photocoagulation) is scarring (e.g. formation of scar tissue).

The condition may for example be retinal detachment. In some embodiments, the condition is a further retinal detachment, in a subject who has previously experienced a retinal detachment. For example, by administering the compound of formula (I) or pharmaceutically acceptable salt thereof following a retinal detachment, membrane formation, traction on the retina, and subsequent further retinal detachment may be prevented. Similarly, by treating or preventing disorders that may arise following eye injury or eye surgery, retinal detachment may be prevented.

Accordingly, in some embodiments, the method is for preventing or reducing the likelihood of reoccurrence of a further retinal detachment, in a subject who has previously experienced a retinal detachment.

In some embodiments, the compound of formula (I) or pharmaceutically acceptable salt thereof, or pharmaceutical composition, is not for use in therapy of eye conditions associated with non-vascular cell proliferation, epithelial to mesenchymal transition, and/or tissue contraction.

As discussed above, there is a need for therapies to treat non-proliferative diabetic retinopathy. For example, with non-proliferative diabetic retinopathy, at present in the early stages regular monitoring may be the only therapy (https://www.aoa.org/patients-and-public/eye-and-vision-problems/glossary-of-eye-and-vision-conditions/diabetic-retinopathy).

Thus, in another aspect, provided herein is a method of preventing and/or treating non-proliferative diabetic retinopathy, comprising,
administering a compound of formula (I):

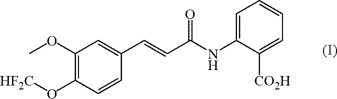

or a pharmaceutically acceptable salt thereof, to the subject.

In some embodiments, the compound or pharmaceutically acceptable salt thereof is administered orally. In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the compound or pharmaceutically acceptable salt thereof is administered at a frequency selected from once per day and twice per day.

In some embodiments, the amount of compound administered is in the range of from 5 mg to 250 mg per day, or the amount of pharmaceutically acceptable salt administered provides an amount of compound in the range of from 5 mg to 250 mg per day.

In some embodiments, a first dose or series of first doses of the compound of formula (I) or a pharmaceutically acceptable salt thereof, is administered to the subject, and
subsequently a second dose or series of second doses of the compound of formula (I) or pharmaceutically acceptable salt thereof, is administered to the subject,
and wherein the amount of compound or pharmaceutically acceptable salt in the or each first dose is greater than the amount of compound or pharmaceutically acceptable salt in the or each second dose.

In some embodiments, the subject is a human.

Also provided herein is a method of preventing and/or treating non-proliferative diabetic retinopathy, comprising,
administering a pharmaceutical composition comprising a) a compound of formula (I):

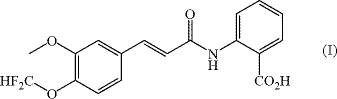

or a pharmaceutically acceptable salt thereof, and b) a pharmaceutically acceptable excipient, to the subject.

In some embodiments, the pharmaceutical composition is administered orally. In some embodiments, following oral administration, the compound has a terminal half-life in the range of from 18 to 30 hours. In some embodiments, following oral administration, the compound has a terminal half-life in the range of from 18 to 24 hours. In some embodiments, following oral administration, a therapeutically effective amount of the compound reaches the eye within 4 hours. In some embodiments, following oral administration, a therapeutically effective amount of the compound reaches one or more of the retina, choroid, retinal pigment epithelium, iris, ciliary body and vitreous humour, within 4 hours.

In some embodiments, the pharmaceutical composition is administered at a frequency selected from once per day and twice per day.

In some embodiments, the amount of pharmaceutical composition administered provides an amount of compound in the range of from 5 mg to 250 mg per day.

In some embodiments, a first dose or series of first doses of a pharmaceutical composition comprising i) and ii) is administered to the subject, and
subsequently a second dose or series of second doses of a pharmaceutical composition comprising i) and ii) is administered to the subject,
and wherein the amount of compound or pharmaceutically acceptable salt in the or each first dose is greater than the amount of compound or pharmaceutically acceptable salt in the or each second dose.

In some embodiments, the subject is a human.

Also provided herein is the use of a compound of formula (I):

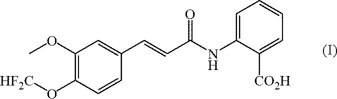

or a pharmaceutically acceptable salt thereof,
for the manufacture of a medicament for the prevention and/or treatment of non-proliferative diabetic retinopathy.

Also provided herein is a compound of formula (I):

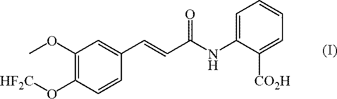

or a pharmaceutically acceptable salt thereof,
for use in the prevention and/or treatment of non-proliferative diabetic retinopathy.

Also provided herein is a pharmaceutical composition comprising i) a compound of formula (I):

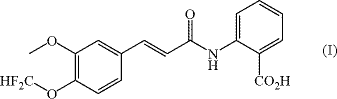

or a pharmaceutically acceptable salt thereof, and ii) a pharmaceutically acceptable excipient, for use in the prevention and/or treatment of non-proliferative diabetic retinopathy.

In some embodiments, the compound of formula (I) or pharmaceutically acceptable salt or pharmaceutical composition is used to prevent or slow progression to proliferative diabetic retinopathy.

### Administration of the Compound

As discussed above, the compound of formula (I), pharmaceutically acceptable salts thereof, and pharmaceutical compositions containing the compound or salt, find use in the therapy of eye conditions.

The methods and uses described herein involve administration of the compound or salt or pharmaceutical composition to a subject. As used herein, the term “subject” refers to any organism susceptible to the relevant disease or condition. For example, the subject may be a mammal. In preferred embodiments, the subject is human. In some preferred embodiments, the subject is male. In other preferred embodiments, the subject is female. In preferred embodiments, the subject is an adult. In other embodiments, the subject is a child.

The compound, salt, or pharmaceutical composition may be administered by any suitable administration route. For example it may be administered intravenously, subcutaneously, intravitreally, or orally.

In some preferred embodiments, the compound of formula (I) or salt or pharmaceutical composition is administered orally. For example, as shown by the examples, it has been found that, upon oral administration to humans, suitable concentrations of the compound are obtained in the plasma.

The compound of formula (I), or salt thereof, or pharmaceutical composition containing the compound or salt, is administered in a therapeutically effective amount. The term “therapeutically effective amount”, as used herein, refers to the compound of Formula (I) or salt thereof, or pharmaceutical composition being administered in an amount and at a dosage interval sufficient to alleviate or prevent to some extent one or more of the symptoms and/or clinical manifestations of the disorder or condition being treated. The result can be for example the reduction and/or alleviation of the signs, symptoms, or causes of a disease or condition.

As shown by the examples, the compound of formula (I) has an unexpectedly long half-life in humans, facilitating an infrequent dosing regime (e.g.once per day or twice per day) and the dosing of comparatively low amounts of compound whilst achieving sustained therapeutic effects (e.g. in the range of from 50 mg/day to 250 mg/day).

Accordingly, in some embodiments, the compound of formula (I) or pharmaceutical salt or pharmaceutical composition is administered not more than twice per day (e.g. not more than twice in a 24 hour period). In some embodiments, the compound of formula (I) or pharmaceutical salt or pharmaceutical composition is administered twice per day. In some embodiments, the compound of formula (I) or pharmaceutical salt or pharmaceutical composition is administered not more than once per day. In some embodiments, the compound of formula (I) or pharmaceutical salt or pharmaceutical composition is administered once per day.

In some aspects of the disclosure, for example where the compound, salt or pharmaceutical composition is administered not more than twice per day, the compound of formula (I) or pharmaceutical salt or pharmaceutical composition is administered such that the amount of compound administered is in the range of from 1 mg to 500 mg per day, or the amount of pharmaceutically acceptable salt administered or the amount of pharmaceutical composition administered provides an amount of compound in the range of from 1 mg to 500 mg per day.

In some embodiments of all aspects of the disclosure, the compound of formula (I) or pharmaceutical salt or pharmaceutical composition is administered such that the amount of compound administered is in the range of from 5 mg to 250 mg per day, or the amount of pharmaceutically acceptable salt administered or the amount of pharmaceutical composition administered provides an amount of compound in the range of from 5 mg to 250 mg per day.

In some embodiments, the compound of formula (I) or pharmaceutical salt or pharmaceutical composition is administered such that the amount of compound administered is in the range of from 1 mg to 150 mg per day, or the amount of pharmaceutically acceptable salt administered or the amount of pharmaceutical composition administered provides an amount of compound in the range of from 1 mg to 150 mg per day.

In some embodiments, the compound of formula (I) or pharmaceutical salt or pharmaceutical composition is administered such that the amount of compound administered is in the range of from 50 mg to 150 mg per day, or the amount of pharmaceutically acceptable salt administered or the amount of pharmaceutical composition administered provides an amount of compound in the range of from 50 mg to 150 mg per day.

In some embodiments, the compound of formula (I) or pharmaceutical salt or pharmaceutical composition is administered such that the amount of compound administered is in the range of from 100 mg to 250 mg per day, or the amount of pharmaceutically acceptable salt administered or the amount of pharmaceutical composition administered provides an amount of compound in the range of from 100 mg to 250 mg per day.

In some embodiments, the compound of formula (I) or pharmaceutical salt or pharmaceutical composition is administered such that the amount of compound administered is in the range of from 100 mg to 150 mg per day, or the amount of pharmaceutically acceptable salt administered or the amount of pharmaceutical composition administered provides an amount of compound in the range of from 100 mg to 150 mg per day.

In some embodiments, the compound of formula (I) or pharmaceutical salt or pharmaceutical composition is administered such that the amount of compound administered is in the range of from 150 mg to 200 mg per day, or the amount of pharmaceutically acceptable salt administered or the amount of pharmaceutical composition administered provides an amount of compound in the range of from 150 mg to 200 mg per day.

In some embodiments, the compound of formula (I) or pharmaceutical salt or pharmaceutical composition is administered such that the amount of compound administered is in the range of from 200 mg to 250 mg per day, or the amount of pharmaceutically acceptable salt administered or the amount of pharmaceutical composition administered provides an amount of compound in the range of from 200 mg to 250 mg per day.

In some embodiments, the compound of formula (I) or pharmaceutical salt or pharmaceutical composition is administered such that the amount of compound administered is in the range of from 150 mg to 300 mg per day, or the amount of pharmaceutically acceptable salt administered or the amount of pharmaceutical composition administered provides an amount of compound in the range of from 150 mg to 300 mg per day.

In some embodiments, the compound of formula (I) or pharmaceutical salt or pharmaceutical composition is administered such that the amount of compound administered is in the range of from 300 mg to 500 mg per day, or the amount of pharmaceutically acceptable salt administered or the amount of pharmaceutical composition administered provides an amount of compound in the range of from 300 mg to 500 mg per day.

In some embodiments, the amount of compound of formula (I), pharmaceutically acceptable salt or pharmaceutical composition administered is such that the amount of compound provided per day is in the range of from 1 to 10 mg, 10 to 20 mg, 20 to 30 mg, 30 to 40 mg, 40 to 50 mg, 50 to 75 mg, 75 to 100 mg, 100 to 125 mg, 125 to 150 mg, 150 to 175 mg, 175 to 200 mg, 200 to 250 mg, 250 to 300 mg, 300 to 350 mg, 350 to 400 mg, 400 to 450 mg, or 450 to 500 mg.

In some embodiments, the amount of compound of formula (I), pharmaceutically acceptable salt or pharmaceutical composition administered is such that the amount of compound provided per day is or is about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg or 500 mg, or within a range defined by any of the aforementioned values.

In some embodiments, a subject is administered an above amount per day in a single dose per day. In some embodiments, a subject is administered an above amount per day in two doses per day, for example two equal doses per day (e.g. a subject is administered 200 mg per day of the compound of formula (I) over two doses and so is administered two doses of a pharmaceutical composition during the day, with each quantity of the pharmaceutical composition administered containing 100 mg of the compound of formula (I)).

In some embodiments, following oral administration, the compound has a terminal half-life in humans in the range of from 12 to 48 hours, or in the range of from 18 to 30 hours, or in the range of from 18 to 24 hours, or in the range of from 21 to 27 hours, or in the range of from 23 to 25 hours, or about 24 hours.

In some embodiments, following oral administration, a therapeutically effective amount of the compound reaches the eye within 6 hours, or within 4 hours, or within 3 hours, or within 2 hours, or within 1 hour following administration.

In some embodiments, following oral administration, a therapeutically effective amount of the compound reaches one or more of the retina, choroid, retinal pigment epithelium, iris, ciliary body and vitreous humour, within 6 hours, or within 4 hours, or within 3 hours, or within 2 hours, or within 1 hour following administration. In some embodiments, following oral administration, a therapeutically effective amount of the compound reaches the retina within 4 hours following administration. In some embodiments, following oral administration, a therapeutically effective amount of the compound reaches the choroid within 4 hours following administration. In some embodiments, following oral administration, a therapeutically effective amount of the compound reaches the retinal pigment epithelium within 4 hours following administration. In some embodiments, following oral administration, a therapeutically effective amount of the compound reaches the iris within 4 hours following administration. In some embodiments, following oral administration, a therapeutically effective amount of the compound reaches the ciliary body within 4 hours following administration. In some embodiments, following oral administration, a therapeutically effective amount of the compound reaches the vitreous humour within 4 hours following administration.

In some embodiments, a staged dosing regime may be used. For example, a first higher loading dose or series of doses (e.g. 2, 3, 4 or 5 doses) of the compound of formula (I) may be administered to provide a therapeutically effective concentration of the compound rapidly, followed by a second dose or series of doses which provide a lower amount of compound of formula (I) which maintains the concentration of the compound at a therapeutically effective concentration.

Thus, in some embodiments, a first dose or series of first doses of a compound of formula (I) or a pharmaceutically acceptable salt thereof, is administered to the subject, and subsequently a second dose or series of second doses of the compound of formula (I) or pharmaceutically acceptable salt thereof, is administered to the subject, wherein the amount of compound or pharmaceutically acceptable salt in the or each first dose is greater than the amount of compound or pharmaceutically acceptable salt in the or each second dose.

In some embodiments, a first dose or series of first doses of a pharmaceutical composition comprising a) a compound of formula (I), or a pharmaceutically acceptable salt thereof, and b) a pharmaceutically acceptable excipient, is administered to the subject, and subsequently a second dose or series of second doses of a pharmaceutical composition comprising a) the compound of formula (I) or a pharmaceutically acceptable salt thereof, and b) a pharmaceutically acceptable excipient, is administered to the subject, wherein the amount of compound or pharmaceutically acceptable salt in the or each first dose is greater than the amount of compound or pharmaceutically acceptable salt in the or each second dose.

In some embodiments, the amount of compound of formula (I), pharmaceutically acceptable salt or pharmaceutical composition administered is such that the amount of compound provided in the or each second dose may be about three quarters, about two thirds, about half, about one third or about one quarter of the amount of compound of formula (I) administered in the or each first dose.

In some embodiments, the amount of compound of formula (I), pharmaceutically acceptable salt or pharmaceutical composition administered is such that the amount of compound provided in the or each second dose may be about 90%, 85%, 80%, 75%, 70%, 65%, 60%, 55%, 50%, 45%, 40%, 35%, 30% or 25% of the amount of compound of formula (I) administered in the or each first dose.

In some embodiments, the amount of compound of formula (I), pharmaceutically acceptable salt or pharmaceutical composition administered is such that the amount of compound provided in the or each first dose is or is about 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg or 500 mg, or within a range defined by any of the aforementioned values.

In some embodiments, the amount of compound of formula (I), pharmaceutically acceptable salt or pharmaceutical composition administered is such that the amount of compound provided in the or each second dose is or is about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, or 200 mg, or within a range defined by any of the aforementioned values.

In some embodiments, the amount of compound of formula (I), pharmaceutically acceptable salt or pharmaceutical composition administered is such that the amount of compound provided in the or each first dose is in the range of from 200 mg to 400 mg and the amount of compound of formula (I) administered in the or each second dose is in the range of from 100 mg to 200 mg.

In some embodiments, the amount of compound of formula (I) administered in the or each first dose is in the range of from 150 mg to 300 mg and the amount of compound of formula (I) administered in the or each second dose is in the range of from 50 mg to 150 mg.

In some embodiments, a single first dose is administered. In some embodiments a series of 2, 3, 4 or 5 first doses is administered.

In some embodiments, a series of second doses administered, for example a series of at least 5, at least 10, at least 15, at least 20, at least 30, at least 40 or at least 50 second doses.

The compound of formula (I) may be administered in any suitable form, for example it may be administered as a salt, or as a free acid. In some embodiments, the free acid of the compound of formula (I) is administered.

Whilst in some embodiments, the compound of formula (I) or salt thereof or pharmaceutical composition may be administered as a sole therapy, in other embodiments the compound or salt or pharmaceutical composition may be administered in combination with a further therapy. In some embodiments, the compound of formula (I) is administered in combination with one or more additional pharmaceutically acceptable agents. The compound of formula (I) or salt thereof, and the one or more further therapeutic/pharmaceutically active agents, may be administered simultaneously, subsequently or separately. For example, they may be administered as part of the same composition, or by administration of separate compositions.

### Pharmaceutical Compositions

Whilst the compound of formula (I) or pharmaceutically acceptable salt thereof may in some embodiments be administered alone, it is more typically administered as part of a pharmaceutical composition or formulation. Thus, the present disclosure also provides a pharmaceutical composition for uses as defined herein, comprising the compound of formula (I) or a pharmaceutically acceptable salt thereof and a pharmaceutically acceptable excipient. The pharmaceutical composition comprises one or more pharmaceutically acceptable diluents, carriers or excipients (collectively referred to herein as “excipient” materials).

The pharmaceutical compositions or formulations may for example be suitable for human medical use. It may also or instead be suitable for veterinary use. The carrier(s) must be pharmaceutically acceptable in the sense of being compatible with the other ingredients of the formulation and not unduly deleterious to the recipient thereof.

Examples of pharmaceutical compositions or formulations include those suitable for oral, parenteral (including intravenous, intravitreal, subcutaneous, intradermal, and intramuscular), inhalation, rectal, intraperitoneal, and topical administration.

The pharmaceutical compositions may conveniently be presented in unit dosage form and may be prepared by methods known in pharmacy. All methods include the step of bringing a compound of formula (I) or a pharmaceutically acceptable salt thereof into association with the excipient or excipients. For example, the compositions may be prepared by uniformly bringing into association the active ingredient with liquid carriers or finely divided solid carriers or both, and then, if necessary, forming the product into the desired formulation.

In some preferred embodiments, the composition is formulated for oral delivery. For example, pharmaceutical compositions of the present disclosure suitable for oral administration may be presented as discrete units such as capsules, sachets, pills or tablets, each containing a predetermined amount of the active ingredient; as a powder or granules, as a solution or a suspension in an aqueous liquid or non-aqueous liquid, for example as elixirs, tinctures, suspensions or syrups; or as an oil-in-water liquid emulsion or a water-in-oil liquid emulsion. A compound of formula (I) may also for example be presented as a bolus, electuary or paste.

A tablet may be made for example by compression or moulding. Compressed tablets may be prepared by compressing in a suitable machine the active ingredient in a free-flowing form such as a powder or granules, optionally mixed with one or more of a binder, lubricant, filler, diluent, surfactant, and a dispersing agent. Moulded tablets may be made for example by moulding in a suitable machine a mixture of the powdered compound moistened with an inert liquid diluent. The tablets may be optionally coated or scored, and may be formulated so as to provide slow or controlled release of the compound of formula (I). The compound of formula (I) can, for example, be administered in a form suitable for immediate release or extended release. Immediate release or extended release can be achieved by the use of suitable pharmaceutical compositions comprising a compound of formula (I) or, particularly in the case of extended release, by the use of devices such as subcutaneous implants or osmotic pumps. A compound of formula (I) may also be administered liposomally.

Exemplary compositions for oral administration include suspensions which can contain, for example, microcrystalline cellulose imparting bulk, alginic acid or sodium alginate as a suspending agent, methylcellulose as a viscosity enhancer, and sweeteners or flavouring agents such as those well known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents, and lubricants such as those known in the art.

Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like.

Disintegrants include without limitation, starch, methylcellulose, agar, bentonite, xanthan gum, and the like.

A compound of formula (I) can also be delivered through the oral cavity by sublingual and/or buccal administration. Moulded tablets, compressed tablets, or freeze-dried tablets are exemplary forms that may be used. Exemplary compositions include those formulating a compound of formula (I) with fast dissolving diluents such as mannitol, lactose, sucrose and/or cyclodextrins. Also included in such formulations may be high molecular weight excipients such as cellulose or polyethylene glycols (PEGs). Such formulations can also include an excipient to aid mucosal adhesion such as hydroxyl propyl cellulose (HPC), hydroxyl propyl methyl cellulose (HPMC), sodium carboxymethylcellulose (SCMC), maleic anhydride copolymer, and agents to control release such as polyacrylic copolymers. Lubricants, glidants, flavours, colouring agents, and stabilisers may also be added. Lubricants used in these dosage forms include sodium stearate, sodium stearyl fumarate, magnesium stearate, and the like.

For oral administration in liquid form, the oral drug components can be combined with any oral, non-toxic, pharmaceutically acceptable inert carrier such as ethanol, glycerol, water, and the like.

In some embodiments, the pharmaceutical compositions of the present disclosure may also include polymeric excipients/additives or carriers, e.g., polyvinylpyrrolidones, derivatised celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations).

In some embodiments, the composition comprising a compound of formula (I) or a pharmaceutically acceptable salt thereof is for administration by parenteral delivery. For example, in one embodiment, the composition may be a sterile, lyophilized composition that is suitable for reconstitution in an aqueous vehicle prior to injection or infusion. For example, the composition may be a reconstituted composition produced by admixing of a solid composition as discussed above with a diluent such as saline or WFI (water for injection). Formulations for parenteral administration include aqueous and non-aqueous sterile injections solutions which may contain anti-oxidants, buffers, bacteriostats, and solutes that render the formulation isotonic with the blood of the intended recipient; and aqueous and non-aqueous sterile suspensions which may include suspending agents and thickening agents.

The formulations may be presented in unit-dose or multi-dose containers, for example, sealed ampoules and vials, and may be stored in a freeze-dried condition requiring only the addition of the sterile liquid carrier, for example saline or water-for-injection, immediately prior to use. Extemporaneous injection solutions and suspensions may be prepared from sterile powders, granules and tablets of the kind previously described.

Exemplary compositions or parenteral administration include injectable solutions or suspensions which can contain, for example, suitable non-toxic, parenterally acceptable diluents or solvents, such as mannitol, 1.3-butanediol, water, Ringer’s solution, an isotonic sodium chloride solution, or other suitable dispersing or wetting and suspending agents, including synthetic mono or diglycerides, and fatty acids, including oleic acid.

For example, in one embodiment, the formulation may be a sterile, lyophilized composition that is suitable for reconstitution in an aqueous vehicle prior to injection. In one embodiment, a formulation suitable for parenteral administration conveniently comprises a sterile aqueous preparation of the compound of formula (I), which may for example be formulated to be isotonic with the blood of the recipient.

In some embodiments, the compound of formula (I) or pharmaceutically acceptable salt thereof is formulated for intravitreal administration, for example it may be present in an intravitreal composition. Intravitreal formulations may for example contain water for injection, a suitable buffer such as citrate, phosphate or sulfate, a surfactant such as polysorbate 20 or polysorbate 80, a tonicity modifier such as sodium chloride, and/or other excipients such as D-mannitol or PEG.

In some embodiments, the compositions comprising a compound of formula (I) or a pharmaceutically acceptable salt thereof may conveniently be presented in unit dosage form.

It should be understood that in addition to the ingredients particularly mentioned above, the formulations may include other agents suitable to the type of formulation in question, for example, those excipients suitable for oral administration may include flavouring agents and/or sweeteners. Examples of pharmaceutical excipients and/or additives suitable for use in the compositions according to the present disclosure are listed in “Remington: The Science & Practice of Pharmacy”, 19^{th} ed., Williams & Williams, (1995), and in the “Physician’s Desk Reference”, 52^{nd} ed., Medical Economics, Montvale, N.J . (1998), and in “Handbook of Pharmaceutical Excipients”, Third Ed., Ed. A. H. Kibbe, Pharmaceutical Press, 2000.

In some embodiments, the compound of formula (I) or pharmaceutically acceptable salt thereof is provided in the form of a pharmaceutical composition in unit dosage form (e.g. such as a tablet, capsule or sachet) which contains an amount in the range of from 1 to 500 mg of the compound, or contains an amount of the salt so as to provide from 1 to 500 mg of the compound. In some embodiments, the unit dosage form contains an amount in the range of from 5 to 200 mg, or from 1 to 150 mg, or from 100 to 250 mg, or from 150 to 300 mg, or from 300 to 500 mg of the compound, or contains an amount of the salt so as to provide an amount in the range of from 5 to 200 mg, or from 1 to 150 mg, or from 100 to 250 mg, or from 150 to 300 mg, or from 300 to 500 mg of the compound. In some embodiments, the unit dosage form contains an amount in the range of from 1 to 10 mg, 10 to 20 mg, 20 to 30 mg, 30 to 40 mg, 40 to 50 mg, 50 to 75 mg, 75 to 100 mg, 100 to 125 mg, 125 to 150 mg, 150 to 175 mg, 175 to 200 mg, 200 to 250 mg, 250 to 300 mg, 300 to 350 mg, 350 to 400 mg, 400 to 450 mg, or 450 to 500 mg of the compound, or contains an amount of the salt so as to provide an amount in the range of from 1 to 10 mg, 10 to 20 mg, 20 to 30 mg, 30 to 40 mg, 40 to 50 mg, 50 to 75 mg, 75 to 100 mg, 100 to 125 mg, 125 to 150 mg, 150 to 175 mg, 175 to 200 mg, 200 to 250 mg, 250 to 300 mg, 300 to 350 mg, 350 to 400 mg, 400 to 450 mg, or 450 to 500 mg of the compound.

In some embodiments, the unit dosage form contains an amount of about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 85 mg, 86 mg, 87 mg, 88 mg, 89 mg, 90 mg, 95 mg, 96 mg, 97 mg, 98 mg, 99 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg or 500 mg of the compound, or contains an amount of the salt so as to provide an amount of about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg or 500 mg of the compound.

The details of specific embodiments described herein are not to be construed as limiting the present disclosure. Various equivalents and modifications may be made without departing from the essence and scope of this disclosure, and it is understood that such equivalent embodiments are part of this disclosure.

### EXAMPLES

The present disclosure is further supported by the following non-limiting examples.

### Example 1: Synthesis of (E)-2-[[3-Methoxy-4-(Difluoromethoxy)Phenyl-1-Oxo-2-Propenyl]Amino]benzoic Acid (The Compound of Formula (I)

The compound of formula (I) may be prepared as described in WO2009/079692A1.

### 2-[(Carboxyacetyl)Amino]Benzoic Acid

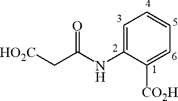 [SMILES: O=C(O)CC(=O)Nc1ccccc1C(=O)O]

Anthranilic acid (300 g, 2.08 mol) was added to a solution of Meldrum’s acid (272 g, 1.98 mol) in toluene (2.0 L). The reaction flask was fitted with a Dean-Stark apparatus and the suspension was heated to reflux for 3 h. The suspension was cooled, filtered, washed with toluene and dried. 2-[(Carboxyacetyl)amino]benzoic acid (381 g, 86%) was obtained as a colourless solid; mp 171-173° C.; ^{δ}H (500 MHz, DMSO-CF₆) 3.45 (br s, 2H, CH₂), 7.16 (t, J 3,4 = 4,5 = 8.0 H z. 1H, H4), 7.59 (t d, J 4,5 = J 5,6 = 8.0, J 3,5 = 1.5 Hz, 1H, H5), 7.97 (dd, J 3,4 = 8.0, J 3,5 = 1.5 Hz, 1H, H3), 8.44 (d, J 5,6 = 8.0 Hz, 1H, H6), 11.27 (s, 1H, NH), 12.83 (br s, 1H, CO₂H), 13.57 (br s, 1H, CO₂H); ^{δ}C (125 MHz, DMSO-CF₆) 45.0, 117.0, 120.3, 123.1, 131.2, 134.1, 140.4, 164.9, 169.1, 169.3; Vₘₐₓ 760, 1234, 1385, 1544, 1684, 1712, 2653, 2964, 3119 cm⁻¹.

### 4-(Difluoromethoxy)-3-methoxybenzaldehyde

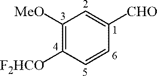 [SMILES: COc1cc(C=O)ccc1OC(F)F]

Methyl chlorodifluoroacetate (1.4 ml, 13 mmol) was added to a suspension of vanillin (1.0 g, 6.6 mmol) and potassium carbonate (2.0 g, 14 mmol) in DMF (10 ml). The suspension was heated to 65-70° C. for 16 h and the suspension was diluted with water. The aqueous phase was extracted with EtOAc and the combined organic fractions were washed with saturated aqueous NaHCO, water, brine, dried and concentrated. The residue was purified by column chromatography, eluting with 10% EtOAc/petrol to give 4-(difluoromethoxy)-3-methoxybenzaldehyde (0.54 g, 41%) as a colourless oil; ^{δ}H (400 MHz, CDCl₃) 3.95 (s, 3H, OCH₃), 6.60 (t, J = 74 Hz, 1H, OCHF₂), 7.30 (d, J 5,6 = 8.0 Hz, 1H, H5), 7.45 (dd, J 5,6 = 8.0, J 2,6 = 2.0 Hz, 1H, H6), 7.50 (d, J 2,6 = 2.0 Hz, 1H, HZ), 9.93 (s, 1H, CHO); ^{δ}C (100 MHz, CDCl₃) 56.2, 110.9, 115.5 (t, J =256 Hz), 121.5, 125.0, 134.5, 144.9, 151.5, 190.8.

### (E)[[3-Methoxy-4-(Difluoromethoxy)Phenyl)-1-OxoPropenyl]Amino]Benzoic Acid

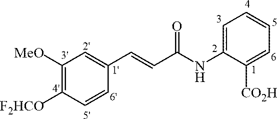 [SMILES: COc1cc(/C=C/C(=O)Nc2ccccc2C(=O)O)ccc1OC(F)F]

Piperidine (0.25 ml, 2.6 mmol) was added to a suspension of 4-(difluoromethoxy)-3-methoxybenzaldehyde (0.52 g, 2.6 mmol) and 2-[(carboxyacetyl)amino]benzoic acid (0.52 mg, 2.6 mmol) in toluene (5.0 ml). The reaction flask was fitted with a Dean-Stark apparatus and heated to reflux for 30 min. The reaction was then cooled to rt and the resulting suspension was filtered and washed with toluene. The piperidinium salt was dissolved in MeOH (5 ml) and water (2 ml) and the solution was acidified with 50% aqueous AcOH. The crude product was collected by filtration and recrystallised from EtOH/water, filtered and washed with water to afford (E)-2-[[3-methoxy-4-(difluoromethoxy)phenyl-1-oxo-2-propenyl]amino]benzoic acid (259 mg, 71%) as a colourless crystalline solid; mp 172-174° C.; ^{δ}H (500 MHz, DMSO-CF₆) 3.90 (s, 3H, OCH3), 6.94 (d, J = 15.6 Hz, 1H, CH=CHCO), 7.12 (t, J = 75 Hz, 1H, OCHF₂), 7.17 (t, J 3,4 = J 4,5 = 8.0 Hz, 1H, H4), 7.20 (d, J = 8.0 Hz, 1H, H5′), 7.32 (dd, J = 8.0, J =2.0 Hz, 1H, H6′), 7.56 (d, J 2′,6′ = 2.0 Hz, 1H, H2′), 7.61 (d, J= 15.6 Hz, 1H, CH=CHCO), 7.62 (dt, J 4,5 = J 5,6 = 8.0, J 3,5 = 1.5 Hz, 1H, H5), 8.00 (dd, J 3,4 = 8.0, J 3,5 = 1.5 Hz, 1H, H3), 8.61 (d, J 5,6 = 8.0 Hz, 1H, H6), 11.33 (s, 1H, NH), 13.60 (br s, 1H, CO₂H); ^{δ}C (125 MHz, DMSO-CF₆) 56.1, 112.3, 114.5, 116.5 (t, J = 256 Hz), 116.8, 120.4, 120.8, 121.4, 122.7, 122.9, 131.1, 132.9, 134.0, 140.6, 140.8, 150.7, 163.7, 169.4; HRMS (ESI) calculated for C₁₈H₁₅F₂NO₅ [M-H] 362.0835, found 362.0839; Vₘₐₓ 1032, 1260, 1586, 1604,1661,2988,3509 cm⁻¹.

### Example 2: Dog and Rat Pharmacokinetic Study With (E)-2-[[3-Methoxy-4-(Difluoromethoxy)Phenyl-1-Oxo-2-Propenyl]Amino]Benzoic Acid

Sprague-Dawley rats were administered a single daily dose of (E)-2-[[3-methoxy-4-(difluoromethoxy)phenyl-1-oxo-2-propenyl]amino]benzoic at 25, 50 or 100 mg/kg for 28 consecutive days. Plasma was collected up to 24 hours post-dose on the first day of dosing. Plasma pharmacokinetic analysis showed that the elimination half-life was 12.9, 13.0, 11.0 hours for the 25, 50 or 100 mg/kg doses, respectively. This rat plasma pharmacokinetic data did not predict a long half-life in humans.

Beagle dogs were administered a single daily dose of (E)-2-[[3-Methoxy-4-(difluoromethoxy)phenyl-1-oxo-2-propenyl]amino]benzoic at 25, 50 or 100 mg/kg for 28 consecutive days. Plasma was collected up to 24 hours post-dose on the first day of dosing. Plasma pharmacokinetic analysis showed that the elimination half-life was 4.3, 4.9 and 4.2 hours for the 25, 50 or 100 mg/kg doses, respectively. This dog plasma pharmacokinetic data did not predict a long half-life in humans.

### Example 3A: Human Clinical Trial Pharmacokinetic Study Using (E)-2-[[3-Methoxy-4-(Difluoromethoxy)Phenyl-1-Oxo-2-Propenyl]Amino]Benzoic Acid

Clinical pharmacokinetic studies showed that (E)-2-[[3-Methoxy-4-(difluoromethoxy)phenyl-1-oxo-2-propenyl]amino]benzoic acid (the compound of formula (I)) has quick absorption and longer than expected elimination in humans.

Six healthy male volunteers in a Phase I clinical trial received a single dose of 50 mg (E)-2-[[3-Methoxy-4-(difluoromethoxy)phenyl-1-oxo-2-propenyl]amino] benzoic acid administered as powder-in-capsule. Blood was collected 10 minutes prior to dosing, and at 15 and 30 minutes and 1, 2, 3, 4, 6, 8, 12, 24, 36, 48, 72 and 96 hours post dose into K₂EDTA tubes. Samples were spun in a refrigerated centrifuge within 1 hour of collection, at 2000 g for 10 minutes. Plasma was collected and frozen on dry ice.

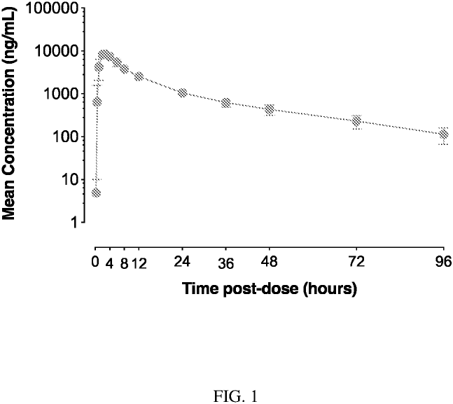

The concentration of (E)-2-[[3-Methoxy-4-(difluoromethoxy)phenyl-1-oxo-2-propenyl]amino]benzoic acid in plasma was determined using a validated LC-MS/MS method. A non-compartmental analysis was performed using WinNonLin software. Nominal doses and sampling times were used. Concentration values below the lower limit of quantitation were treated as zero for pharmacokinetic analysis. Results are shown in Table 1A. The mean pharmacokinetic profile in humans is also shown in FIG. 1.

Pharmacokinetic data for a comparator compound Tranilast following 200 mg oral dosage are also shown. See, e.g., Charng M, Ding P Y, Chuang M, et al. Pharmacokinetic Properties of Tranilast in Chinese People. Journal of Food and Drug Analysis, Vol. 10, No. 3, 2002, Pages 135-138. Tranilast is approved for use in an amount of 3 × 100 mg/day.

TABLE 1A

Non-compartmental plasma PK parameters following oral dosing of (E)-2-[[3-Methoxy-4-(difluoromethoxy)phenyl-1-oxo-2-propenyl]amino]benzoic acid or the comparator Tranilast in humans

| | (E)-2-[[3-Methoxy-4-(difluoromethoxy)phenyl-1-oxo-2-propenyl]amino]benzoic acid 50 mg | Tranilast 200 mg (comparative example; data from Charng et al.) |
|---|---|---|
| Cₘₐₓ (ng/mL) | 8,742 | 42,000 |
| AUC_{0-inf} (ng·h/mL) | 112,785 | 412,000 |
| Dose normalised Cₘₐₓ (ng/mL) | 175 | 210 |
| Dose normalised AUC_{0-inf} ng·h/mL | 2,256 | 2,060 |
| Tₘₐₓ (h) | 2.5 | 2.8 |
| T _{½} (h) | 24.0 | 7.5 |

Pharmacokinetic calculations of plasma PK parameters of (E)-2-[[3-Methoxy-4-(difluoromethoxy)phenyl-1-oxo-2-propenyl]amino]benzoic acid indicated a long elimination half-life of 24 hours, in contrast to what was predicted from the corresponding animal experiments. The half-life of (E)-2-[[3-Methoxy-4-(difluoromethoxy)phenyl-1-oxo-2-propenyl]amino]benzoic acid was also much longer than that of the comparator (half-life of 7.5 hours). At the same time, (E)-2-[[3-Methoxy-4-(difluoromethoxy)phenyl-1-oxo-2-propenyl]amino]benzoic acid was absorbed as rapidly as the comparator Tranilast, with Tₘₐₓ of 2.5 hours.

Given these pharmacokinetic parameters, (E)-2-[[3-Methoxy-4-(difluoromethoxy)phenyl-1-oxo-2-propenyl]amino]benzoic acid can unexpectedly be dosed less frequently and/or in lower daily dosage amounts than Tranilast.

Moreover, (E)-2-[[3-Methoxy-4-(difluoromethoxy)phenyl-1-oxo-2-propenyl]amino]benzoic acid had an unexpectedly long half life in humans based on that predicted from dog and rat pharmacokinetic data.

### Example 3B: Human Clinical Trial Pharmacokinetic Study Using (E)-2-[[3-Methoxy-4-(Difluoromethoxy)Phenyl-1-Oxo-2-Propenyl]Amino]Benzoic Acid

Clinical pharmacokinetic studies were also conducted on volunteers who received a single dose or multiple doses of 200 mg (E)-2-[[3-Methoxy-4-(difluoromethoxy)phenyl-1-oxo-2-propenyl]amino] benzoic acid administered as powder-in-capsule. Blood was collected 10 minutes prior to dosing, and at 15 and 30 minutes and 1, 2, 3, 4, 6, 8, 12, 24, 36, 48, 72 and 96 hours post dose into K₂EDTA tubes. Samples were spun in a refrigerated centrifuge within 1 hour of collection, at 2000 g for 10 minutes. Plasma was collected and frozen on dry ice.

The concentration of (E)-2-[[3-Methoxy-4-(difluoromethoxy)phenyl-1-oxo-2-propenyl]amino]benzoic acid in plasma was determined using a validated LC-MS/MS method. A non-compartmental analysis was performed using WinNonLin software. Nominal doses and sampling times were used. Concentration values below the lower limit of quantitation were treated as zero for pharmacokinetic analysis. Results are shown in Table 1. The mean pharmacokinetic profile in humans is also shown in FIG. 1.

TABLE 1B

Non-compartmental plasma PK parameters following oral dosing of (E)-2-[[3-Methoxy-4-(difluoromethoxy)phenyl-1-oxo-2-propenyl]amino]benzoic acid or the comparator Tranilast in humans

| | (E)-2-[[3-Methoxy-4-(difluoromethoxy)phenyl-1-oxo-2-propenyl]amino]benzoic acid | (E)-2-[[3-Methoxy-4-(difluoromethoxy)phenyl-1-oxo-2-propenyl]amino]benzoic acid | Tranilast 200 mg (comparative example; data from Charng et al., Day 1, single dose) |
|---|---|---|---|
| | 200 mg (Day 1, single dose) | 200 mg (Day 14, multiple dose) | |
| Cₘₐₓ (ng/mL) | 32,783 | 67,633 | 42,000 |
| AUC_{0-inf} (ng-h/mL) | 390,281 | 3,774,727 | 412,000 |
| Dose normalised Cₘₐₓ (ng/mL) | 164 | 338 | 210 |
| Dose normalised AUC_{0-inf} ng·h/mL | 1951 | 18,874 | 2,060 |
| Tₘₐₓ (h) | 3.0 | 2.5 | 2.8 |
| T _{½} (h) | 9.6 | 23.2 | 7.5 |

The data shows that (E)-2-[[3-Methoxy-4-(difluoromethoxy)phenyl-1-oxo-2-propenyl]amino]benzoic acid accumulates in the plasma with multiple dosing such that the steady state exposure after 14 days of dosing is significantly higher, as evidence by a two-fold increase in Cmax compared with a single dose. This effect is unexpected, as animal pharmacokinetic data did not show accumulation even with 91 days of dosing in rats or dogs.

### Example 4: Pharmacokinetic Modelling of (E)-2-[[3-Methoxy-4-(Difluoromethoxy)Phenyl-1-Oxo-2-Propenyl]Amino]Benzoic Acid

Population pharmacokinetic modeling and simulation of (E)-2-[[3-Methoxy-4-(difluoromethoxy)phenyl-1-oxo-2-propenyl]amino]benzoic acid oral dosing in humans was carried out using pre-clinical plasma pharmacokinetic data from dogs. The predicted Cₘₐₓ and AUC₀₋₂₄ values from the modelling indicated that high dosage levels would be required to reach required levels of exposure.

The modelling did not accurately predict the steady state exposure of (E)-2-[[3-Methoxy-4-(difluoromethoxy)phenyl-1-oxo-2-propenyl]amino]benzoic acid based on Cₘₐₓ and AUC₀₋₂₄. The corresponding data from the human Phase I clinical study is also included below, showing a much greater than predicted Cₘₐₓ and AUC₀₋₂₄ at 50 mg dosing.

| Dose | Data Source | C_{max,} ₛₛ (ng/mL) | AUC_{0-24 hrs,} ₛₛ (ng*h/mL) |
|---|---|---|---|
| 50 mg | PK Modelling | 1,530 | 9,882 |
| | Data from Phase I (after 14 days dosing) | 6,562 | 73,955 |

### Example 5: Animal Pharmacokinetic Study With (E)-2-[[3-Methoxy-4-(Difluoromethoxy)Phenyl-1-Oxo-2-Propenyl]Amino]Benzoic Acid

Pharmacokinetic studies in rabbits and rats showed rapid distribution of (E)-2-[[3-Methoxy-4-(difluoromethoxy)phenyl-1-oxo-2-propenyl]amino]benzoic acid (the compound of formula (I)) to the eye.

Male Dutch Belted pigmented rabbits were administered a once daily dose of 50 or 100 mg/kg of (E)-2-[[3-Methoxy-4-(difluoromethoxy)phenyl-1-oxo-2-propenyl]amino]benzoic acid by oral gavage for 8 days. The oral formulation was prepared in 1% carboxymethyl cellulose. Blood was collected into tubes containing K₂EDTA from two animals/dose/time point at 0 (predose), 1, 2, 4, 8, and 24 hours after the last dose on Study Day 8. Immediately following blood collection, animals were sacrificed via overdose of sodium pentobarbital. Blood samples were placed in a chilled cryorack until centrifuged to obtain plasma, which was stored at approximately -70° C. At the time of sacrifice for all animals, both eyes were enucleated, the aqueous humor was collected and discarded, and each eye was then flash frozen in liquid nitrogen for 15 to 20 seconds. The enucleated eye was placed on dry ice or stored at approximately -70° C. for at least two hours. Within approximately 5 days, the choroid-RPE, iris-ciliary body, retina and vitreous humor were collected from the right and left eye. The ocular tissues were rinsed with saline and blotted dry, as appropriate, weighed, and placed on dry ice. Sample analysis was performed using a qualified LC-MS/MS method to ascertain the concentration of (E)-2-[[3-Methoxy-4-(difluoromethoxy)phenyl-1-oxo-2-propenyl]amino]benzoic acid in each tissue matrix. Non-compartmental analysis (Gibaldi and Perrier, 1982) was applied to the individual and mean concentration data. Nominal doses and sampling times were used. Concentration values below the lower limit of quantitation were treated as zero for pharmacokinetic analysis. The plasma and ocular steady state non-compartmental pharmacokinetic parameters for repeated oral administration of 50 or 100 mg/kg on Day 8 are summarized in Table 2 and further below.

TABLE 2

Steady state non-compartmental PK parameters for pigmented rabbits after 8 consecutive single daily oral doses of (E)-2-[[3-Methoxy-4-(difluoromethoxy)phenyl-1-oxo-2-propenyl]amino]benzoic acid

| | Plasma | | Vitreous Humour | |
|---|---|---|---|---|
| Target Dose (mg/kg/day) | 50 | 100 | 50 | 100 |
| Cₘₐₓ (ng/mL) | 15200 | 42000 | 3.3 | 11.5 |
| Tₘₐₓ (h) | 1 | 2 | 1 | 4 |

| | | | | | | |
|---|---|---|---|---|---|---|
| | Retina | | Iris Ciliary Body | | Choroid-RPE | |
| Target Dose (mg/kg/day) | 50 | 100 | 50 | 100 | 50 | 100 |
| Cₘₐₓ (ng/g) | 623 | 2370 | 2510 | 8620 | 3320 | 12300 |
| Tₘₐₓ (h) | 1 | 4 | 1 | 2 | 1 | 2 |

(E)-2-[[3-Methoxy-4-(difluoromethoxy)phenyl-1-oxo-2-propenyl]amino]benzoic acid was rapidly absorbed with plasma Tₘₐₓ of 1 and 2 hours for 50 and 100 mg/kg, respectively. Similarly, (E)-2-[[3-Methoxy-4-(difluoromethoxy)phenyl-1-oxo-2-propenyl]amino]benzoic acid was rapidly distributed to ocular tissues with Tₘₐₓ between 1-4 hours for all analyzed tissues. For both doses, the maximum concentration was observed in the choroid-RPE then ICB, retina, and vitreous humor. Exposure (Cₘₐₓ) was greatest in the choroid-RPE for both doses (3320 and 12300 ng/g for 50 and 100 mg/kg, respectively). (E)-2-[[3-Methoxy-4-(difluoromethoxy)phenyl-1-oxo-2-propenyl]amino] benzoic acid could not be detected in plasma or ocular tissue by 24 hours-post dose for either dose, in rabbits. The results indicated rapid distribution to ocular tissues and greatest exposure (as indicated by Cₘₐₓ) in the posterior of the eye (choroid/RPE).

Male Sprague Dawley rats were administered a single dose of 50 mg/kg of (E)-2-[[3-Methoxy-4-(difluoromethoxy)phenyl-1-oxo-2-propenyl]amino]benzoic acid by oral gavage. The oral formulation was prepared in 1% carboxymethyl cellulose. Samples were collected from 3 animals/timepoint at 1, 2, 4, 8 and 24 hours after dosing. Whole blood was collected from the tail vein in conscious rats into polypropylene tubes containing heparin. Blood samples were centrifuged immediately (3000 rpm, for 10 minutes) and supernatant plasma removed and snap frozen in liquid N2. After blood collection, animals were euthanised with pentobarbitone (120 mg/kg), eyes were enucleated and retina were weighed, collected and snap frozen in liquid nitrogen. Blood was centrifuged at 3000 rpm for 10 mins at 4° C. and plasma was collected and snap frozen in liquid N2. Plasma and retinal concentrations of (E)-2-[[3-Methoxy-4-(difluoromethoxy)phenyl-1-oxo-2-propenyl]amino]benzoic acid were determined using a validated LC-MS/MS method. Cₘₐₓ and Tₘₐₓ was estimated using GraphPad prism software. The preliminary results are summarised below.

The estimated maximum concentration in plasma was 35,134 ng/mL at 1 hour-post dose. Similarly, (E)-2-[[3-Methoxy-4-(difluoromethoxy)phenyl-1-oxo-2-propenyl]amino]benzoic acid was rapidly distributed to the retina, with estimated maximal exposure at 4 hours and estimated Cₘₐₓ of 792.5 ng/g. (E)-2-[[3-Methoxy-4-(difluoromethoxy)phenyl-1-oxo-2-propenyl]amino]benzoic acid was still detected in the plasma and retina at 24-hours post-dose.

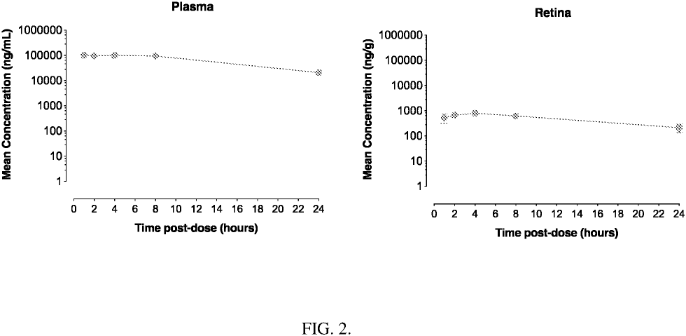

FIG. 2 also shows the pharmacokinetic profile of plasma and retina of male SD rats after oral administration of a single dose of 50 mg/kg of (E)-2-[[3-methoxy-4-(difluoromethoxy)phenyl-1-oxo-2-propenyl]amino]benzoic acid.

Collectively, this data indicates that (E)-2-[[3-Methoxy-4-(difluoromethoxy)phenyl-1-oxo-2-propenyl]amino]benzoic was rapidly distributed to relevant ocular tissues in both species.

Although the present disclosure has been described with reference to embodiments and examples, it should be understood that numerous and various modifications can be made without departing from the spirit of the present disclosure. Accordingly, the present disclosure is limited only by the following claims.

All references cited herein, including patents, patent applications, papers, text books, and the like, and the references cited herein, to the extent that they are not already, are hereby incorporated by reference in their entirety. In the event that one or more of the incorporated literature and similar materials differ from or contradict this application, including but not limited to defined terms, term usage, described techniques, or the like, this application controls.

What is claimed is:

1. A method of preventing, treating, reducing the severity of and/or reducing the likelihood of reoccurrence of an eye condition in a subject, comprising,
administering a compound of formula (I): 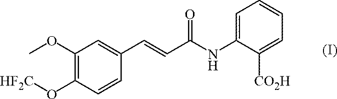
or a pharmaceutically acceptable salt thereof, to the subject,
wherein the compound or pharmaceutically acceptable salt thereof is administered at a frequency selected from once per day and twice per day.

2. A method as claimed in claim 1, wherein the compound or pharmaceutically acceptable salt thereof is administered orally.

3. A method as claimed in claim 2 wherein, following oral administration, the compound has a terminal half-life in the range of from 18 to 30 hours.

4. A method as claimed in claim 3 wherein, following oral administration, the compound has a terminal half-life in the range of from 18 to 24 hours.

5. A method as claimed in any of claims 2 to 4 wherein, following oral administration, a therapeutically effective amount of the compound reaches the eye within 4 hours.

6. A method as claimed in claim 5 wherein, following oral administration, a therapeutically effective amount of the compound reaches one or more of the retina, choroid, retinal pigment epithelium, iris, ciliary body and vitreous humour, within 4 hours.

7. A method as claimed in any of claims 1 to 6, wherein the compound or pharmaceutically acceptable salt thereof is administered twice per day.

8. A method as claimed in any of claims 1 to 6, wherein the compound or pharmaceutically acceptable salt thereof is administered once per day.

9. A method as claimed in any of claims 1 to 8, wherein the subject is a human.

10. A method of preventing, treating, reducing the severity of and/or reducing the likelihood of reoccurrence of an eye condition in a subject, comprising,
administering a pharmaceutical composition comprising i) a compound of formula (I): 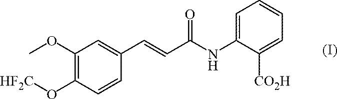
or a pharmaceutically acceptable salt thereof, and ii) a pharmaceutically acceptable excipient, to the subject,
wherein the pharmaceutical composition is administered at a frequency selected from once per day and twice per day.

11. A method as claimed in claim 10, wherein the pharmaceutical composition is administered orally.

12. A method as claimed in claim 11 wherein, following oral administration, the compound has a terminal half-life in the range of from 18 to 30 hours.

13. A method as claimed in claim 12 wherein, following oral administration, the compound has a terminal half-life in the range of from 18 to 24 hours.

14. A method as claimed in any of claims 11 to 13 wherein, following oral administration, a therapeutically effective amount of the compound reaches the eye within 4 hours.

15. A method as claimed in claim 14 wherein, following oral administration, a therapeutically effective amount of the compound reaches one or more of the retina, choroid, retinal pigment epithelium, iris, ciliary body and vitreous humour, within 4 hours.

16. A method as claimed in any of claims 11 to 15, wherein the pharmaceutical composition is administered twice per day.

17. A method as claimed in any of claims 11 to 15, wherein the pharmaceutical composition is administered once per day.

18. A method as claimed in any of claims 11 to 17, wherein the subject is a human.

19. A method as claimed in any of claims 1 to 18, wherein the eye condition is an eye condition associated with at least one of inflammation and vascular proliferation.

20. A method as claimed in claim 19, wherein the eye condition is selected from the group consisting of diabetic retinopathy and age-related macular degeneration.

21. A method as claimed in claim 20, wherein the eye condition is proliferative diabetic retinopathy.

22. A method as claimed in claim 20, wherein the eye condition is non-proliferative diabetic retinopathy.

23. A method as claimed in claim 20, wherein the eye condition is wet age-related macular degeneration.

24. A method as claimed in any of claims 1 to 18, wherein the eye condition is an eye condition which is associated with at least one of the following: non-vascular cell proliferation, epithelial to mesenchymal transition, and tissue contraction.

25. A method as claimed in claim 24, wherein the eye condition is proliferative vitreoretinopathy.

26. A method as claimed in any of claims 1 to 18, wherein the method is for preventing or reducing the likelihood of reoccurrence of a further retinal detachment, in a subject who has previously experienced a retinal detachment.

27. Use of a compound of formula (I): 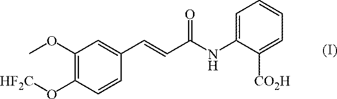
or a pharmaceutically acceptable salt thereof,
for the manufacture of a medicament for the prevention, treatment, reduction of severity of and/or reduction of likelihood of reoccurrence of an eye condition in a subject,
wherein the compound or pharmaceutically acceptable salt thereof is administered to the subject at a frequency selected from once per day and twice per day.

28. A compound of formula (I): 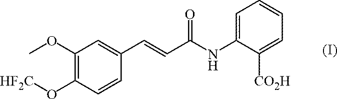
or a pharmaceutically acceptable salt thereof, for use in the prevention, treatment, reduction of severity of and/or reduction of likelihood of reoccurrence of an eye condition in a subject, wherein the compound or pharmaceutically acceptable salt thereof is administered to the subject at a frequency selected from once per day and twice per day.

29. A pharmaceutical composition comprising i) a compound of formula (I): 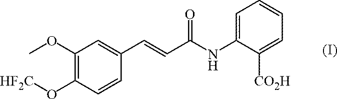
or a pharmaceutically acceptable salt thereof, and ii) a pharmaceutically acceptable excipient, for use in the prevention, treatment, reduction of severity of and/or reduction of likelihood or reoccurrence of an eye condition in a subject, wherein the pharmaceutical composition is administered to the subject at a frequency selected from once per day and twice per day.

30. Use as claimed in claim 27, a compound for use as claimed in claim 28, or a pharmaceutical composition for use as claimed in claim 29, wherein following oral administration, the compound has a terminal half-life in the range of from 18 to 30 hours.

31. Use as claimed in claim 27, a compound for use as claimed in claim 28, or a pharmaceutical composition for use as claimed in claim 29, wherein, following oral administration, the compound has a terminal half-life in the range of from 18 to 24 hours.

32. A method of preventing, treating, reducing the severity of and/or reducing the likelihood of reoccurrence of an eye condition in a subject, comprising,
administering a compound of formula (I): 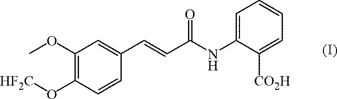
or a pharmaceutically acceptable salt thereof, to the subject,
wherein the amount of compound administered is in the range of from 5 mg to 250 mg per day, or the amount of pharmaceutically acceptable salt administered provides an amount of compound in the range of from 5 mg to 250 mg per day.

33. A method as claimed in claim 32, wherein the compound or pharmaceutically acceptable salt thereof is administered orally.

34. A method as claimed in claim 33 wherein, following oral administration, the compound has a terminal half-life in the range of from 18 to 30 hours.

35. A method as claimed in claim 34 wherein, following oral administration, the compound has a terminal half-life in the range of from 18 to 24 hours.

36. A method as claimed in any of claims 33 to 35 wherein, following oral administration, a therapeutically effective amount of the compound reaches the eye within 4 hours.

37. A method as claimed in claim 36 wherein, following oral administration, a therapeutically effective amount of the compound reaches one or more of the retina, choroid, retinal pigment epithelium, iris, ciliary body and vitreous humour, within 4 hours.

38. A method as claimed in any of claims 32 to 37, wherein the amount of compound administered is in the range of from 100 mg to 250 mg per day, or the amount of pharmaceutically acceptable salt administered provides an amount of compound in the range of from 100 mg to 250 mg per day.

39. A method as claimed in any of claims 32 to 38, wherein the subject is a human.

40. A method of preventing, treating, reducing the severity of and/or reducing the likelihood of reoccurrence of an eye condition in a subject, comprising,
administering a pharmaceutical composition comprising i) a compound of formula (I): 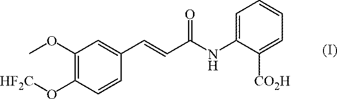
or a pharmaceutically acceptable salt thereof, and ii) a pharmaceutically acceptable excipient, to the subject,
wherein the amount of pharmaceutical composition administered provides an amount of compound in the range of from 5 mg to 250 mg per day.

41. A method as claimed in claim 40, wherein the pharmaceutical composition is administered orally.

42. A method as claimed in claim 41 wherein, following oral administration, the compound has a terminal half-life in the range of from 18 to 30 hours.

43. A method as claimed in claim 42 wherein, following oral administration, the compound has a terminal half-life in the range of from 18 to 24 hours.

44. A method as claimed in any of claims 41 to 43 wherein, following oral administration, a therapeutically effective amount of the compound reaches the eye within 4 hours.

45. A method as claimed in claim 44 wherein, following oral administration, a therapeutically effective amount of the compound reaches one or more of the retina, choroid, retinal pigment epithelium, iris, ciliary body and vitreous humour, within 4 hours.

46. A method as claimed in any of claims 40 to 45, wherein the amount of pharmaceutical composition administered provides an amount of compound in the range of from 100 mg to 250 mg per day.

47. A method as claimed in any of claims 40 to 46, wherein the subject is a human.

48. A method as claimed in any of claims 32 to 47, wherein the eye condition is an eye condition associated with one or more of inflammation and vascular proliferation.

49. A method as claimed in claim 48, wherein the eye condition is selected from the group consisting of diabetic retinopathy and age-related macular degeneration.

50. A method as claimed in claim 49, wherein the eye condition is proliferative diabetic retinopathy.

51. A method as claimed in claim 49, wherein the eye condition is non-proliferative diabetic retinopathy.

52. A method as claimed in claim 49, wherein the eye condition is wet age-related macular degeneration.

53. A method as claimed in any of claims 32 to 47, wherein the eye condition is an eye condition which is associated with at least one of non-vascular cell proliferation, epithelial to mesenchymal transition and tissue contraction.

54. A method as claimed in claim 53, wherein the eye condition is proliferative vitreoretinopathy.

55. A method as claimed in any of claims 32 to 47, wherein the method is for preventing or reducing the likelihood of reoccurrence of a further retinal detachment, in a subject who has previously experienced a retinal detachment.

56. Use of a compound of formula (I): 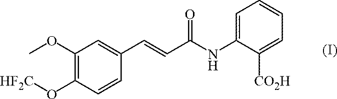
or a pharmaceutically acceptable salt thereof,
for the manufacture of a medicament for the prevention, treatment, reduction of severity of and/or reduction of likelihood of reoccurrence of an eye condition in a subject,
wherein the amount of compound administered is in the range of from 5 mg to 250 mg per day, or the amount of pharmaceutically acceptable salt administered provides an amount of compound in the range of from 5 mg to 250 mg per day.

57. A compound of formula (I): 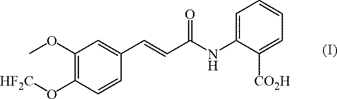
or a pharmaceutically acceptable salt thereof,
for use in the prevention, treatment, reduction of severity of and/or reduction of likelihood of reoccurrence of an eye condition in a subject,
wherein the amount of compound administered is in the range of from 5 mg to 250 mg per day, or the amount of pharmaceutically acceptable salt administered provides an amount of compound in the range of from 5 mg to 250 mg per day.

58. A pharmaceutical composition comprising i) a compound of formula (I): 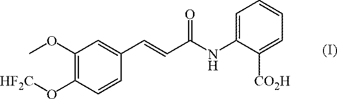
or a pharmaceutically acceptable salt thereof, and ii) a pharmaceutically acceptable excipient, for use in the prevention, treatment, reduction of severity of and/or reduction of likelihood of reoccurrence of an eye condition in a subject,
wherein the amount of pharmaceutical composition administered provides an amount of compound in the range of from 5 mg to 250 mg per day.

59. Use as claimed in claim 56, a compound for use as claimed in claim 57, or a pharmaceutical composition for use as claimed in claim 58, wherein following oral administration, the compound has a terminal half-life in the range of from 18 to 30 hours.

60. Use as claimed in claim 56, a compound for use as claimed in claim 57, or a pharmaceutical composition for use as claimed in claim 58, wherein, following oral administration, the compound has a terminal half-life in the range of from 18 to 24 hours.

61. A method of preventing, treating, reducing the severity of and/or reducing the likelihood of reoccurrence oft an eye condition in a subject, comprising,
administering a first dose or series of first doses of a compound of formula (I): 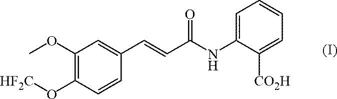
or a pharmaceutically acceptable salt thereof, to the subject, and
subsequently administering a second dose or series of second doses of the compound of formula (I) or pharmaceutically acceptable salt thereof, to the subject,
wherein the amount of compound or pharmaceutically acceptable salt in the or each first dose is greater than the amount of compound or pharmaceutically acceptable salt in the or each second dose.

62. A method as claimed in claim 61, wherein the compound or pharmaceutically acceptable salt thereof is administered orally.

63. A method as claimed in claim 62 wherein, following oral administration, the compound has a terminal half-life in the range of from 18 to 30 hours.

64. A method as claimed in claim 63 wherein, following oral administration, the compound has a terminal half-life in the range of from 18 to 24 hours.

65. A method as claimed in any of claims 62 to 64 wherein, following oral administration, a therapeutically effective amount of the compound reaches the eye within 4 hours.

66. A method as claimed in claim 65 wherein, following oral administration, a therapeutically effective amount of the compound reaches one or more of the retina, choroid, retinal pigment epithelium, iris, ciliary body and vitreous humour, within 4 hours.

67. A method as claimed in any of claims 61 to 66, wherein the subject is a human.

68. A method of preventing, treating, reducing the severity of and/or reducing the likelihood of reoccurrence of an eye condition in a subject, comprising,
administering a first dose or series of first doses of a pharmaceutical composition comprising a) a compound of formula (I): 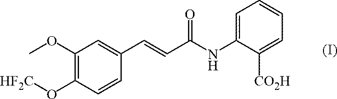
or a pharmaceutically acceptable salt thereof, and b) a pharmaceutically acceptable excipient, to the subject, and
subsequently administering a second dose or series of second doses of a pharmaceutical composition comprising a) the compound of formula (I) or a pharmaceutically acceptable salt thereof, and b) a pharmaceutically acceptable excipient, to the subject,
wherein the amount of compound or pharmaceutically acceptable salt in the or each first dose is greater than the amount of compound or pharmaceutically acceptable salt in the or each second dose.

69. A method as claimed in claim 68, wherein the pharmaceutical composition is administered orally.

70. A method as claimed in claim 69 wherein, following oral administration, the compound has a terminal half-life in the range of from 18 to 30 hours.

71. A method as claimed in claim 70 wherein, following oral administration, the compound has a terminal half-life in the range of from 18 to 24 hours.

72. A method as claimed in any of claims 69 to 71 wherein, following oral administration, a therapeutically effective amount of the compound reaches the eye within 4 hours.

73. A method as claimed in claim 72 wherein, following oral administration, a therapeutically effective amount of the compound reaches one or more of the retina, choroid, retinal pigment epithelium, iris, ciliary body and vitreous humour, within 4 hours.

74. A method as claimed in any of claims 68 to 73, wherein the subject is a human.

75. A method as claimed in any of claims 61 to 74, wherein the eye condition is an eye condition associated with one or more of inflammation and vascular proliferation.

76. A method as claimed in claim 75, wherein the eye condition is selected from the group consisting of diabetic retinopathy and age-related macular degeneration.

77. A method as claimed in claim 76, wherein the eye condition is proliferative diabetic retinopathy.

78. A method as claimed in claim 76, wherein the eye condition is non-proliferative diabetic retinopathy.

79. A method as claimed in claim 76, wherein the eye condition is wet age-related macular degeneration.

80. A method as claimed in any of claims 61 to 74, wherein the eye condition is an eye condition which is associated with non-vascular cell proliferation, epithelial to mesenchymal transition, and/or tissue contraction.

81. A method as claimed in claim 80, wherein the eye condition is proliferative vitreoretinopathy.

82. A method as claimed in any of claims 61 to 74, wherein the method is for preventing or reducing the likelihood of reoccurrence of a further retinal detachment, in a subject who has previously experienced a retinal detachment.

83. Use of a compound of formula (I): 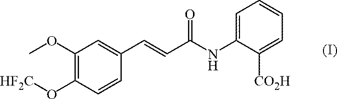
or a pharmaceutically acceptable salt thereof,
for the manufacture of a medicament for the prevention, treatment, reduction of severity of and/or reduction of likelihood of reoccurrence of an eye condition in a subject,
wherein a first dose or series of first doses of the compound of formula (I) or a pharmaceutically acceptable salt thereof, is administered to the subject, and
subsequently a second dose or series of second doses of the compound of formula (I) or pharmaceutically acceptable salt thereof, is administered to the subject,
and wherein the amount of compound or pharmaceutically acceptable salt in the or each first dose is greater than the amount of compound or pharmaceutically acceptable salt in the or each second dose.

84. A compound of formula (I): 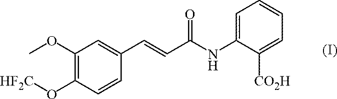
or a pharmaceutically acceptable salt thereof,
for use in the prevention, treatment, reduction of severity of and/or reduction of likelihood of reoccurrence of an eye condition in a subject,
wherein a first dose or series of first doses of the compound of formula (I) or a pharmaceutically acceptable salt thereof, is administered to the subject, and
subsequently a second dose or series of second doses of the compound of formula (I) or pharmaceutically acceptable salt thereof, is administered to the subject,
and wherein the amount of compound or pharmaceutically acceptable salt in the or each first dose is greater than the amount of compound or pharmaceutically acceptable salt in the or each second dose.

85. A pharmaceutical composition comprising i) a compound of formula (I): 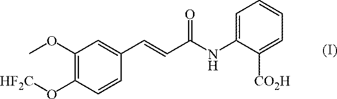
or a pharmaceutically acceptable salt thereof, and ii) a pharmaceutically acceptable excipient, for use in the prevention, treatment, reduction of severity of and/or reduction of likelihood of reoocurrence of an eye condition in a subject,
wherein a first dose or series of first doses of a pharmaceutical composition comprising i) and ii) is administered to the subject, and
subsequently a second dose or series of second doses of a pharmaceutical composition comprising i) and ii) is administered to the subject,
and wherein the amount of compound or pharmaceutically acceptable salt in the or each first dose is greater than the amount of compound or pharmaceutically acceptable salt in the or each second dose.

86. Use as claimed in claim 83, a compound for use as claimed in claim 84, or a pharmaceutical composition for use as claimed in claim 85, wherein following oral administration, the compound has a terminal half-life in the range of from 18 to 30 hours.

87. Use as claimed in claim 83, a compound for use as claimed in claim 84, or a pharmaceutical composition for use as claimed in claim 85, wherein, following oral administration, the compound has a terminal half-life in the range of from 18 to 24 hours.
